(19) United States
(12) Patent Application Publication
(10) Pub. No.: US 2023/0310532 A1
(43) Pub. Date: Oct. 5, 2023

(54) FOOD EXTRACTS, METHODS OF EXTRACTION AND COMPOSITIONS THEREOF

(71) Applicant: National University of Singapore, Singapore (SG)

(72) Inventors: Zhixuan SONG, Singapore (SG); Dejian HUANG, Singapore (SG); Yi Hui Joanne TOY, Singapore (SG); Yi LIN, Singapore (SG)

(21) Appl. No.: 17/995,938

(22) PCT Filed: Apr. 13, 2021
(86) PCT No.: PCT/SG2021/050204

§ 371 Date: Oct. 11, 2022

(30) Foreign Application Priority Data
Apr. 13, 2020 (SG) 10202003345S

(51) Int. Cl. A61K 36/39; A61K 31/7048; A61K 9/48

(52) U.S. Cl. CPC A61K 36/39; A61K 9/4858; A61K 31/7048; A61K 2236/35; A61K 2236/39; A61K 2236/53; A61K 2236/55; A61K 2236/333

(57) ABSTRACT

The present invention relates, in general terms, to a method of extracting resin glycoside from a plant selected from the Convolvulaceae family. The resin glycosides can be extracted from Ipomoea batatas and/or Ipomoea aquatica. The present invention also relates to the extracts and edible compositions thereof and its use in weight loss and weight management.

### TECHNICAL FIELD

The present invention relates, in general terms, to a method of extracting resin glycoside from a plant selected from the Convolvulaceae family. The present invention also relates to an edible composition and its use in weight loss and weight management.

### BACKGROUND

The weight loss and weight management diet market is expected to garner $422.8 billion by 2020, registering a CAGR of 9.1% during the forecast period of 2015-2020. In particular, the total U.S. weight loss market grew at an estimated 4.1% in 2018, from $69.8 billion to $72.7 billion. The total US market is forecast to grow 2.6% annually through 2023.

The large market size is in part due to the understanding that obesity can lead to health issues and consequently higher medical costs and insurance premium. In particular, statistics have shown that the incidences of obesity-related disorders such as diabetes, cardiovascular diseases, and others have considerably increased in past few years. Moreover, a linear time trend forecast suggests that by 2030, around 51% of the world’s population would be affected by obesity. Owing to the increasing health disorders due to overweight and obesity, consumers have started adopting various weight loss and weight management diets including better-for-you food and beverages, weight loss supplements, and others. As a result of this increased consumption of weight loss and weight management products, this market is witnessing growing projection in terms of revenue and the trend is expected to continue over the forecast period.

For example, prescription obesity drugs are currently available to patients. For example, a patient can use Olestra. However, while Olestra can reduces calorie intake from fat as a synthetic fat replacer, it also inhibits absorption of some essential vitamins in food, and may cause abdominal cramps and loose stools. Because of these side effects, it was “honored’ to be one of the 50 worst inventions by TIME magazine.

Another anti-obesity drug on the market is Orlistat. Orlistat is a synthetic lipase inhibitor, is notorious for its gastrointestinal side effects and acute kidney injury may also occur during the treatment. Studies have shown that this drug has a meagre effect on weight, Common side effects of Orlistat are oily spotting on underwear, flatulence, urgent bowel movements, fatty or oily stools, increased number of bowel movements, abdominal pain or discomfort, and inability to control stool (incontinence). Serious side effects include pre-cancerous lesions of the colon (aberrant crypt foci), liver damage and pancreatitis.

No new anti-obesity drugs are expected to enter the market and gain FDA approval before 2022.

To combat obesity, governments are also considering various policies. For example, the Singapore government has recently announced that advertisements for high-sugar drinks are banned in Singapore. Nutrition labels will also be placed on such drinks, with “unhealthy” labels for drinks with medium-to-high sugar content. A tax on sugar is also in consideration.

Surveys have also shown that dieters want to eat “clean”, eliminating preservatives, GMOs, artificial flavours and sweeteners, and this is forcing producers of low-calorie (diet) frozen entrées and diet companies in general to reformulate their foods.

It would be desirable to overcome or ameliorate at least one of the above-described problems, or at least to provide a useful alternative.

### SUMMARY

The present invention is predicated on the understanding that some natural products such as theaflavins can act as lipase inhibitors. Theaflavins are polyphenols that are formed from the condensation of flavan-3-ols in tea leaves during the enzymatic oxidation. The inventors have found that other natural compounds (other than from tea leaves) can act as lipid inhibitors. In this regard, the inventors have found a way to extract such compounds from other types of natural plant ingredients. It was also found that depending on the extraction method, the lipid inhibitory function can be improved. These extracted compounds can be formulated into a food product, a supplement and/or a medicament for use in anti-obesity purpose in controlling digestion and absorption. As the extracted compounds are from a natural plant source, the perceived health and safety value in consumers is high.

The present invention provides an extract comprising at least one resin glycoside selected from the group consisting of Batataoside I, Batataoside II, Batatoside III, Pescaprein XXVII, Batatoside H, Batatoside F, a resin glycoside of Formula (I),

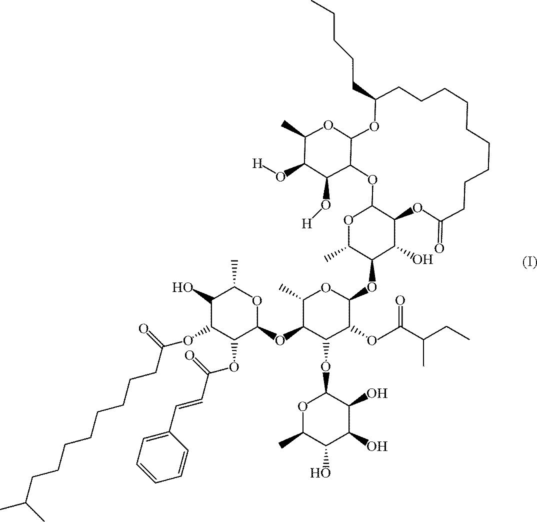

a resin glycoside of Formula (II),

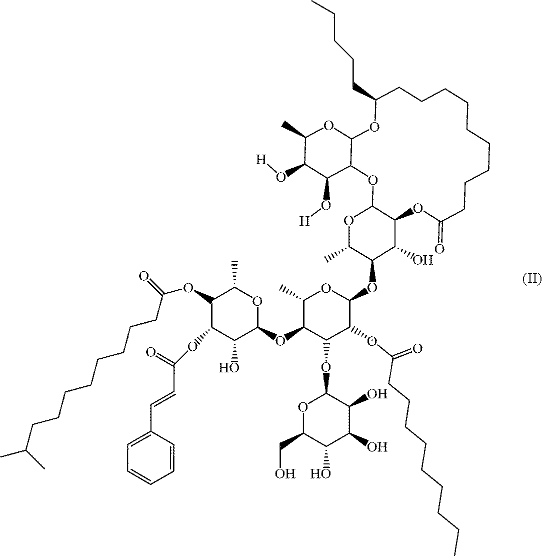

wherein the resin glycoside is extracted from a plant selected from Ipomoea batatas (sweet potato).

In some embodiments, when Batataoside I is present, the concentration is about 0.5 mg/g to about 500 mg/g relative to the extract;
when Batataoside II is present, the concentration is about 0.1 mg/g to about 5 mg/g relative to the extract;
when Batataoside III is present, the concentration is about 0.1 mg/g to about 10 mg/g relative to the extract;
when Batatoside F is present, the concentration is about 2 mg/g to about 40 mg/g relative to the extract;
when Batatoside H is present, the concentration is about 2 mg/g to about 8 mg/g relative to the extract;
when Pescaprein XXVII is present, the concentration is about 0.1 mg/g to about 5 mg/g relative to the extract;
when resin glycoside of Formula (I) is present, the concentration is about 0.1 mg/g to about 4 mg/g relative to the extract; and
when resin glycoside of Formula (II) is present, the concentration is about 10 mg/g to about 500 mg/g relative to the extract.

The resin glycoside can be extracted from all parts of plant, including roots, tuber, leaves, seeds, stems, and flowers. In some embodiments, the resin glycosides are extracted from tuber, leaf, latex, and stem of Ipomoea batatas (sweet potato).

In some embodiments, the extract comprises a mixture of at least 2 resin glycosides.

In some embodiments, the at least one resin glycoside is Batatoside I and resin glycoside of Formula (I).

In some embodiments, the extract has a lipase inhibition activity IC₅₀ value of less than 15 µg/mL.

The present invention provides an extract comprising at least one resin glycoside selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII),

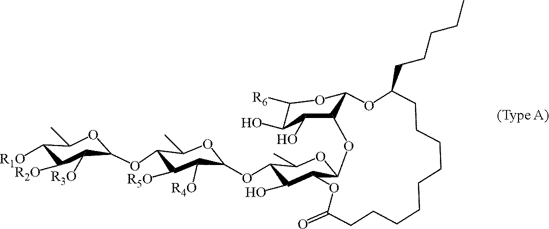

wherein Formula (III) is when R₁ is n-dodecanoyl, R₂ is cinnamoyl, R₃ is 2-methylbutanoyl, R₄ is H, R₅ is rhamnopyranosyl, and R₆ is methyl;
wherein Formula (IV) is when R₁ is isobutanoyl, R₂ is H, R₃ is H, R₄ is n-octanoyl, R₅ is glucopyranosyl, and R₆ is methyl;
wherein Formula (V) is when R₁ is n-octanoyl, R₂ is n-octanoyl, R₃ is H, R₄ is 2-methylbutanoyl, R₅ is n-octanoyl, and R₆ is methyl;
wherein Formula (VI) is when R₁ is n-dodecanoyl, R₂ is H, R₃ is H, R₄ is H, R₅ is n-octanoyl, and R₆ is methyl;
wherein Formula (VII) is when R₁ is n-octanoyl, R₂ is H, R₃ is n-octanoyl, R₄ is 2-methylbutanoyl, R₅ is glucopyranosyl, and R₆ is methyl; and
wherein the resin glycoside is extracted from a plant selected from Ipomoea aquatica (kangkong).

In some embodiments, the resin glycosides are extracted from a seed, leaf or stem of Ipomoea aquatica (kangkong).

The present invention also provides an edible composition, comprising:

at least one resin glycoside extracted from a plant selected from Ipomoea batatas (sweet potato) and Ipomoea aquatica (kangkong) as disclosed herein.

In some embodiments, the edible composition further comprises a component selected from protein, fiber, polyphenolic compound, lipid or a combination thereof.

In some embodiments, the edible composition is provided as a capsule.

In some embodiments, the edible composition is for use in weight loss or weight management.

The present invention also discloses a pharmaceutical composition comprising at least one resin glycoside extracted from a plant selected from Ipomoea batatas (sweet potato) and Ipomoea aquatica (kangkong) as disclosed herein, or a pharmaceutically acceptable salt, solvate or isomer thereof, optionally in combination with a pharmaceutically acceptable carrier, excipient or diluent.

The present invention discloses a method of extracting resin glycoside from a plant selected from Ipomoea batatas (sweet potato) and Ipomoea aquatica (kangkong) as disclosed herein, comprising:
a) subjecting the plant to a solvent extraction process for obtaining a crude extract;
b) purifying the crude extract for obtaining a partially purified extract; and
c) further purifying the partially purified extract for obtaining the resin glycoside.

In some embodiments, the step of subjecting the plant to a solvent extraction process (step a) comprises homogenising the plant in a solvent and liquid-liquid extracting the resin glycoside as a crude extract.

In some embodiments, the step of purifying the crude extract (step b) comprises fractionating the crude extract.

In some embodiments, the step of purifying the crude extract (step b) comprises eluting the crude extract through a liquid chromatography column with a predetermined mobile phase.

In some embodiments, the mobile phase is a gradient of hexane:ethyl acetate (hexane:EtOAc; 1:1) to pure ethyl acetate, followed by pure methanol.

In other embodiments, the mobile phase is a gradient of dichloromethane:methanol (DCM:MeOH; 7:1) to pure MeOH.

In other embodiments, the mobile phase is a gradient of H₂O:MeOH (100:0) to pure MeOH.

In some embodiments, the further purifying step (step c) comprises fractionating the partially purified extract.

In some embodiments, the further purifying step (step c) comprises eluting the partially purified extract through a liquid chromatography column with a predetermined mobile phase.

In some embodiments, the further purifying step (step c) comprises eluting the partially purified extract through a liquid chromatography column with a mobile phase having a gradient of MeOH-H₂O (90:10) to MeOH-H₂O (100:0).

In some embodiments, the further purifying step (step c) further comprises eluting a faction from the liquid chromatography column with a mobile phase having a gradient of MeOH-H₂O (90:10) to MeOH-H₂O (100:0) with acetonitrile (ACN) or with ACN-MeOH (80:20).

The present invention also discloses a method of treating a disease or disorder associated with excessive body fat in a subject in need thereof, comprising administering resin glycoside from a plant selected from the Convolvulaceae family as disclosed herein.

In some embodiments, the disease or disorder associated with excessive body fat is selected from obesity, overweight, and metabolic syndrome.

### BRIEF DESCRIPTION OF THE DRAWINGS

Embodiments of the present invention will now be described, by way of non-limiting example, with reference to the drawings in which:

FIG. 1 is a representative plot of PL inhibitory activity assay using pNPP as substrate and Orlistat as inhibitor. (A) Kinetic readings of optical density at 410 nm under six Orlistat concentrations. (B) Dose-response curve of PL inhibition vs Orlistat concentration;

FIG. 2 is a HPLC chromatographic plot from a first round of fractionation (F5.1.23);

FIG. 3 is a HPLC chromatographic plot from a second round of fractionation (F5.1.23.34);

FIG. 4 illustrates a HPLC chromatographic plot of fraction 8 (F5.1.23.8) after a third round of fractionation;

FIG. 5 illustrates a HPLC chromatographic plot of fraction 10 (F5.1.23.10) after a third round of fractionation;

FIG. 6 illustrates a Pancreatic Lipase (PL) inhibitory assay for sweet potato leaves;

FIG. 7 illustrates a HPLC chromatogram analysis of CC4, overlapped with relative lipase activity in bold red line;

FIG. 8 illustrates fraction a HPLC chromatographic plot of CC4.14.1 after a third round of fractionation;

FIG. 9 illustrates lipase inhibition activity of baby kangkong and kangkong seeds;

FIG. 10 is a schematic diagram of an electrospray setup; and

FIG. 11 illustrates a morphology of the encapsulated particles with extract inside.

### DETAILED DESCRIPTION

The present invention is predicated on the discovery that resin glycosides in the convolvulaceae (morning glory) family can act as lipase inhibitor. It was found that the compound can suppress the digestion and absorption of fats and restrict energy intake. For example, and in particular, it was found that sweet potato and kangkong, two common vegetables in Singapore, showed relative high content of resin glycosides and accordingly lipase inhibition ability. The lipase inhibition ability of an extract was further found to be influenced by the method of extraction, and in this regard, the inventors have found a particular combination of extraction methods is advantageous for providing an extract with good lipase inhibition ability. Further, food formulation comprising resin glycoside extracted from morning glory family vegetables were formulated and which are capable of retarding fat digestion. The vegetable extracts and the formulations can be for use in anti-obesity applications by controlling digestion and absorption. It is believed that this invention can fill the vacancy of functional food products focusing on lipid digestion modulation and body weight management.

As will be shown herein, ethanol extract of sweet potato tubers showed pancreatic lipase (PL) inhibition activity, with IC₅₀ of 33 µg/mL. By further purifying the extract, several resin glycosides including Batatoside I, Batatoside II, Batatoside III and Pescaprein XXVII can be isolated with IC₅₀ among 4.9 to 6.7 µM. Resin glycoside contributed as the major active compound in sweet potato tubers extract for its lipase inhibition activity, and sweet potato tubers extract would be a potent ingredient for functional food with weight-losing function.

Accordingly, the present invention discloses a method of extracting at least one resin glycoside from a plant selected from the Convolvulaceae family, comprising:
a) subjecting the plant to a solvent extraction process for obtaining a crude extract;
b) purifying the crude syrup for obtaining a partially purified extract; and
c) further purifying the partially purified extract for obtaining the resin glycoside.

Convolvulaceae, known commonly as the bindweed or morning glory family, is a family of about 60 genera and more than 1,650 species of mostly herbaceous vines, but also trees, shrubs and herbs, and also including the sweet potato and a few other food tubers. Convolvulaceae can be recognized by their funnel-shaped, radially symmetrical corolla; the floral formula for the family has five sepals, five fused petals, five epipetalous stamens (stamens fused to the petals), and a two-part syncarpous and superior gynoecium. The stems of these plants are usually winding, hence their Latin name (from convolvere, “to wind”). The leaves are simple and alternate, without stipules. In parasitic Cuscuta they are reduced to scales. The fruit can be a capsule, berry, or nut, all containing only two seeds per one locule (one ovule/ovary). The leaves and starchy, tuberous roots of some species are used as foodstuffs (e.g. sweet potato and water spinach), and the seeds are exploited for their medicinal value as purgatives. Some species contain ergoline alkaloids. Members of the family are well known as showy garden plants (e.g. morning glory) and as troublesome weeds (e.g. bindweed and dodder), while Humbertia madagascariensis is a medium-sized tree.

In some embodiments, the plant is selected from a Ipomoea genus in the flowering plant Convolvulaceae family.

Ipomoea is the largest genus in the flowering plant family Convolvulaceae, with over 500 species. It is a large and diverse group with common names including morning glory, water convolvulus or kangkung, sweet potato, bindweed, or moonflower. The most widespread common name is morning glories, but there are also species in related genera bearing the same common name. Those formerly separated in Calonyction are called moonflowers. The generic name is derived from the Greek meaning “woodworm” and “resembling.” It refers to their twining habit. The genus occurs throughout the tropical and subtropical regions of the world, and comprises annual and perennial herbaceous plants, lianas, shrubs and small trees; most of the species are twining climbing plants.

In some embodiments, the plant is selected from Ipomoea batatas (sweet potato) and Ipomoea aquatica (kangkong or kangkung). In other embodiments, the plant is selected from Ipomoea abrupta R.Br., Ipomoea alba L. - moon vine, Ipomoea alpina Rendle, Ipomoea amnicola Morong - red-center morning glory, Ipomoea aquatica Forssk. - water spinach, water morning glory, water convolvulus, “Chinese spinach”, “swamp cabbage”, Ipomoea arborescens G.Don, Ipomoea aristolochiaefolia, Ipomoea asarifolia, Ipomoea barbatisepala A.Gray, Ipomoea batatas (L.) Lam. - sweet potato, “tuberous morning glory”, Ipomoea batatoides Benth., Ipomoea bona-nox, Ipomoea cairica - Coast morning glory, Cairo morning glory, mile-a-minute vine, Messina creeper, railroad creeper, Ipomoea calobra F.Muell., Ipomoea capillacea (Kunth) G.Don, Ipomoea camea - pink morning glory, canudo-de-pita (Brazil), Ipomoea chrysocalyx D.F.Austin, Ipomoea coccinea - red morning glory, redstar, Mexican morning glory, Ipomoea cordatotriloba L. - little violet morning glory, Ipomoea cordatotriloba var. torreyana - purple bindweed, Ipomoea cordifolia Carey ex Voight - heart-leaved morning glory, Ipomoea costata - rock morning glory, bush potato, Ipomoea costellata Torr. - crest-ribbed morning glory, Ipomoea cristulata Hallier f. - trans-Pecos morning glory, Ipomoea cynanchifolia (Meisn.) Mart., Ipomoea daturaefolia Meisn., Ipomoea demerariana Choisy (= I. phyllomega), Ipomoea diversifolia R.Br., Ipomoea dumetorum Willd. ex Roemer & J.A.Schultes - railwaycreeper, Ipomoea eggersiana Peter, Ipomoea eggersii (House) D.Austin - Egger’s morning glory, Ipomoea eriocarpa R.Br., Ipomoea ghika, Ipomoea gracilis R.Br., Ipomoea graminea R.Br., Ipomoea halierca, Ipomoea hederacea - ivy-leaved morning glory, Ipomoea hederifolia - scarlet morning glory, scarlet creeper, star ipomoea, trompillo (= I. coccinea Sessé & Moc.), Ipomoea holubii Baker, Ipomoea horrida Huber, Ipomoea horsfalliae - Lady Doorly’s morning glory, cardinal creeper, Prince Kuhio vine, Ipomoea imperati (Vahl) Griseb, Ipomoea incisa R.Br., Ipomoea indica- oceanblue morning glory, blue morning glory, blue dawn flower, koali awa (Hawaii), Ipomoea jalapa (L.) Pursh., Ipomoea krugii Urban - Krug’s white morning glory, Ipomoea lacunosa L. - whitestar potato, whitestar, Ipomoea leptophylla - bush morning glory, bush moonflower, manroot, Ipomoea leucantha[verification needed] Jacq. (non Webb ex Hook., Desv. ex Ham.), Ipomoea lindheimeri Gray - Lindheimer’s morning glory, Ipomoea littoralis Blume - white-flowered beach morning glory, Ipomoea lobata (Cerv.) Thell. - fire vine, Spanish flag, Ipomoea longifolia Benth. - pink-throated morning glory, Ipomoea macrantha, Ipomoea macrorhiza Michx. - large-rooted morning glory, Ipomoea marginata (Desr.) Verdc., Ipomoea mauritiana Jacq. - giant potato, kiribadu ala, likam (Hawaii), Ipomoea meyeri (Spreng.) G.Don - Meyer’s morning glory, Ipomoea microdactyla Griseb. -calcareous morning glory, Ipomoea × multifida - ‘Cardinal Climber’ (I. coccinea × I. quamoclit), Ipomoea nil - white-edged morning glory, ivy morning glory, Japanese morning glory, Ipomoea obscura - obscure morning glory, small white morning glory, Ipomoea ochracea (Lindl.) G.Don - fence morning glory, Ipomoea oenotherae Hallier f., Ipomoea pandurata - wild potato vine, big-rooted morning glory, man-of-the-earth, manroot, Ipomoea pes-caprae (L.) R.Br. - beach morning glory, “goat’s foot”, Ipomoea pes-caprae ssp. brasiliensis - salsa-da-praia (Brazil), Ipomoea plebeia R.Br., Ipomoea plummerae Gray -Huachuca Mountain morning glory, Ipomoea polymorpha Roem. & Schult. (= I. heterophylla R.Br.), Ipomoea prismatosyphon Welw., Ipomoea pubescens Lam. - silky morning glory (= I. heterophylla Ortega), Ipomoea pulcherrima, Ipomoea purga (Wender.) Hayne - Vera Cruz jalap (= I. jalapa auct. non L.), Ipomoea purpurea - common morning glory, purple morning glory, tall morning glory, Ipomoea quamoclit - cypress vine, cypressvine morning glory, cardinal creeper, cardinal vine, star glory, “hummingbird vine”, Ipomoea racemigera F.Muell. & Tate, Ipomoea repanda Jacq. - bejuco Colorado, Ipomoea repens, Ipomoea rubens Choisy (= I. fragans), Ipomoea rupicola House - cliff morning glory, Ipomoea sagittata Poir. - saltmarsh morning glory, Ipomoea setifera Poir. - bejuco de Puerco, Ipomoea setosa Ker Gawl. - Brazilian morning glory, Ipomoea shumardiana (Torr.) Shinners - narrow-leaved morning glory, Ipomoea simplex Thunb., Ipomoea simulans -Tampico jalap, purga de Sierra Gorda, Ipomoea × sloteri - cardinal climber, Ipomoea steudelii Millsp. - Steudel’s morning glory, Ipomoea stolonifera, Ipomoea tastensis Brandegee from Baja California Sur, Ipomoea temascaltepecensis Wilkin, Ipomoea tenuiloba Torr. - spiderleaf, Ipomoea tenuirostris, Ipomoea tenuissima Choisy - rockland morning glory, Ipomoea ternifolia Cav. - triple-leaved morning glory, Ipomoea thurberi Gray - Thurber’s morning glory, Ipomoea tricolor Cav. - Mexican morning glory, tlitliltzin (Nahuatl), badoh negro, Ipomoea trifida Wild ancestor of the sweet potato, Ipomoea triloba - littlebell, Aiea morning glory, Ipomoea tuberculate, Ipomoea tuberosa L. - Hawaiian woodrose, Ipomoea tuboides O.Deg. & van Ooststr. - Hawaii morning glory, Ipomoea turbinata Lag. - lilacbell, Ipomoea velutina R.Br., Ipomoea violacea L. - beach moonflower, sea moonflower, and Ipomoea wrightii - Wright’s morning glory.

The resin glycoside can be extracted from all parts of plant, including roots, tuber, leaves, seeds, stems, and flowers. In some embodiments, the resin glycoside is extracted from a tuber (thickened underground part of a stem or rhizome), leaf, latex and stem of sweet potato, or a seed, leaf or stem of Kangkong (Kangkung).

Resin glycosides are also known as purgative ingredients; i.e. having a laxative effect. Resin glycoside can be considered to be a structural derivative of carbohydrate, and are high molecular weight oligosaccharide derivatives of hydroxylated fatty acids. Depending on their solubility in ether, these are roughly classified into two groups—jalapin (soluble) and convolvulin (insoluble). Almost all jalapins have a common intramolecular macrocyclic ester structure. These are composed of 1 mol of oligoglycoside of hydroxyl fatty acid (glycosidic acid) partially acylated by some organic acids at the sugar moiety, some examples of which are ester-type dimers. On the other hand, convolvulin is regarded as an oligomer of a variety of acylated glycosides.

Without wanting to be bound by theory, the inventors have found, for the first time, that resin glycosides can act as lipase inhibitors. Resin glycosides are not absorbed into the bloodstream. When they act as lipase inhibitors, they bind to lipase enzymes in the intestine, thus preventing the hydrolysis of dietary triglycerides into monoglycerides and fatty acids. This then reduces the absorption of dietary fat. It is believed that due to the ester bonds, lipase active site can be bounded to the ester bonds and thus hydrolyzed it in a slow manner, causing lipase inhibition.

In some embodiments, the resin glycoside is selected from the group consisting of Batataoside I, Batataoside II, Batatoside III, Pescaprein XXVII, Batatoside F and Batatoside H and a combination thereof. In other embodiments, the resin glycosides are selected from Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII and a combination thereof. These resin glycosides can, for example, be extracted from sweet potato and/or Kangkong. The chemical structures of these compounds are as follows:

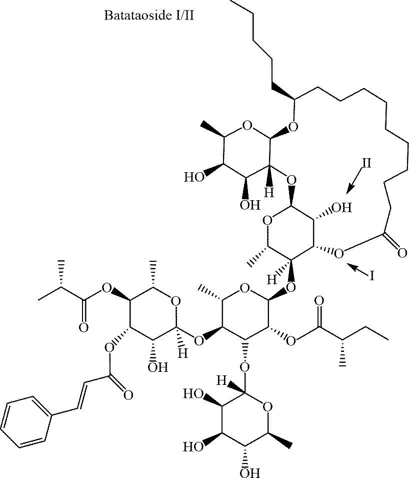

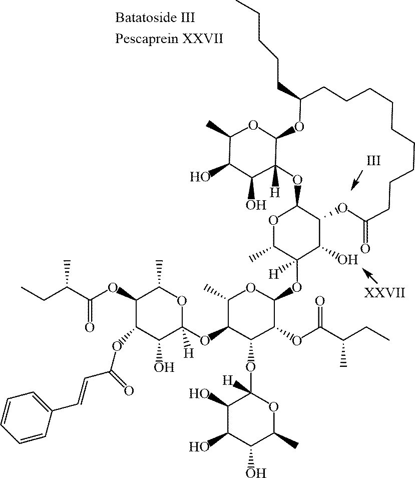

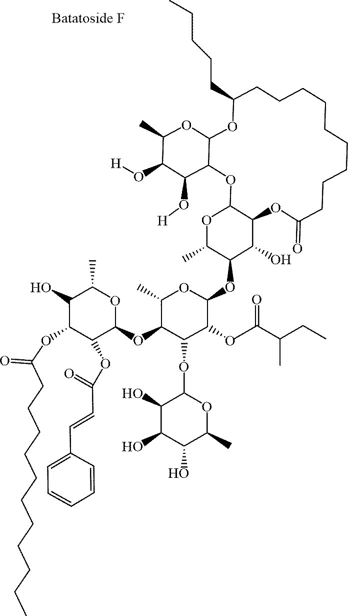

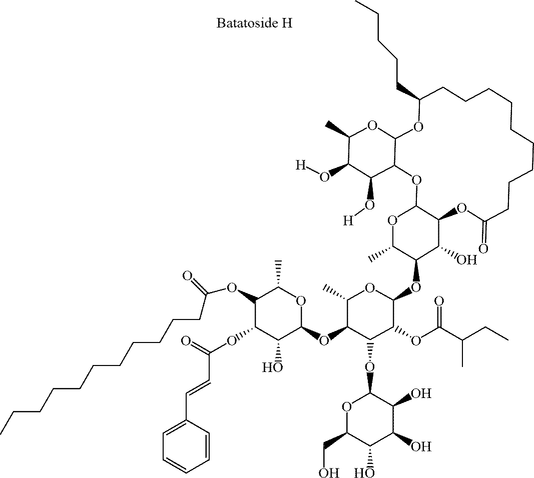

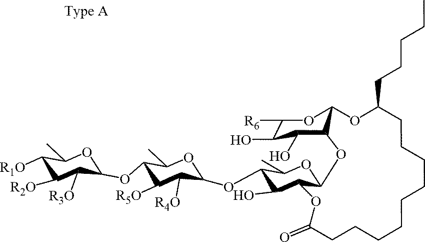

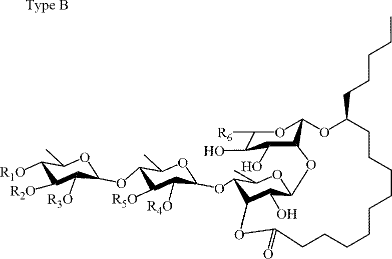

| | | | | | | | | |
|---|---|---|---|---|---|---|---|---|
| Compound | Molecular formula | R₁ | R₂ | R₃ | R₄ | R₅ | R₆ | Type |
| Batatoside F | C₇₂H₁₁₆O₂₅ | H | Dodeca | Cna | Mba | Rha | CH₃ | A |
| Batatoside E | C₇₂H₁₁₆O₂₅ | Dodeca | H | Cna | Mba | Rha | CH₃ | A |
| Batatoside D | C₇₂H₁₁₆O₂₅ | Dodeca | Cna | H | Mba | Rha | CH₃ | A |
| Aquaterin VI | C₆₂H₁₀₈O₂₄ | H | Octa | H | Octa | Rha | CH₃ | A |
| Aquaterin V | C₆₂H₁₀₈O₂₄ | Octa | H | H | Octa | Rha | CH₃ | A |
| Aquaterin XI | C₅₆H₉₈O₂₀ | Octa | H | H | H | Octa | CH₃ | A |
| Aquaterin II | C₅₉H₁₀₂O₂₄ | Octa | H | H | Mba | Rha | CH₃ | A |
| Stoloniferin VIII | C₅₉H₁₀₂O₂₄ | Mba | H | H | Octa | Rha | CH₃ | A |
| Aquaterin XIV | C₆₇H₁₁₆O₂₅ | Octa | H | Octa | Mba | Rha | CH₃ | A |
| Batatoside C | C₇₂H₁₁₆O₂₅ | H | Dodeca | Cna | Mba | Rha | CH₃ | B |
| Batatinoside I | C₇₂H₁₁₆O₂₅ | Dodeca | Cna | H | Mba | Rha | CH₃ | B |
| Aquaterin VII | C₆₂H₁₀₈O₂₄ | Octa | H | H | Octa | Rha | CH₃ | B |
| Aquaterin III | C₅₉H₁₀₂O₂₄ | Octa | H | H | Mba | Rha | CH₃ | B |
| Murucoidin XVIII | C₅₉H₁₀₂O₂₄ | Mba | H | H | Octa | Rha | CH₃ | B |
| Aquaterin XIII | C₆₇H₁₁₆O₂₅ | Octa | Octa | H | Mba | Rha | CH₃ | B |
| Aquaterin XII | C₆₇H₁₁₆O₂₅ | Octa | H | Octa | Mba | Rha | CH₃ | B |

Mba: 2-methylbutanoyl,
Dodeca: n-dodecanoyl,
Octa: n-octanoyl,
Deca: n-decanoyl,
Cna: cinnamoyl,
Rha: Rhamnopyranosyl,
Glu: Glucopyranosyl;
Iba: Isobutanoyl

In some embodiments, the method of extracting at least one resin glycoside comprises a resin glycoside of Formula (I):

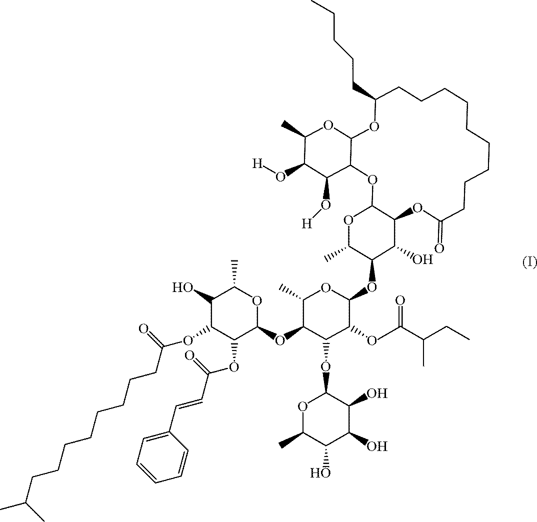

Resin glycoside of Formula (I) can, for example, be extracted from sweet potato tuber.

In some embodiments, the method of extracting at least one resin glycoside comprises a resin glycoside of Formula (II):

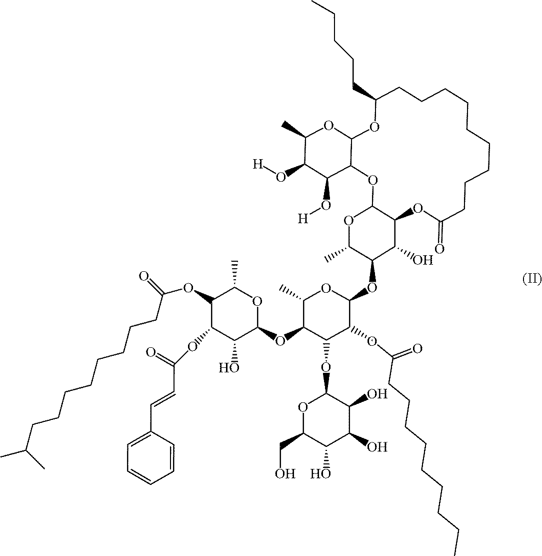

Resin glycoside of Formula (II) can, for example, be extracted from sweet potato leaves.

In some embodiments, the method of extracting at least one resin glycoside comprises a Type A resin glycoside of Formula (III), Formula (IV), Formula (V), Formula (VI), and Formula (VII):

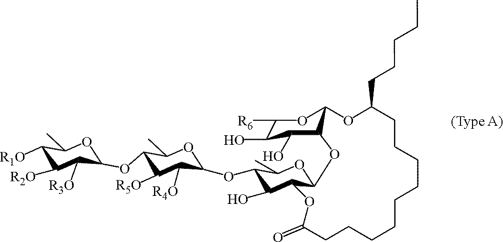

wherein

| | | | | | | | | |
|---|---|---|---|---|---|---|---|---|
| Compound | Molecular formula | R₁ | R₂ | R₃ | R₄ | R₅ | R₆ | Type |
| Formula (III) | C₆₇H₁₁₆O₂₆ | Dodeca | Cna | Mba | H | Rha | CH₃ | A |
| Formula (IV) | C₅₈H₁₀₀O₂₅ | Iba | H | H | Octa | Glu | CH₃ | A |
| Formula (V) | C₆₈H₁₂₀O₂₂ | Octa | Octa | H | Mba | Octa | CH₃ | A |
| Formula (VI) | C₅₈H₁₀₂O₂₀ | Deca | H | H | H | Octa | CH₃ | A |
| Formula (VII) | C₆₇H₁₁₆O₂₆ | Octa | H | Octa | Mba | Glu | CH₃ | A |

Mba: 2-methylbutanoyl, Dodeca: n-dodecanoyl, Octa: n-octanoyl, Deca: n-decanoyl, Cna: cinnamoyl, Rha: Rhamnopyranosyl, Glu: Glucopyranosyl; Iba: Isobutanoyl

The method of extracting resin glycoside comprises subjecting the plant to a solvent extraction process for obtaining a crude extract. In some embodiments, the step of subjecting the plant to a solvent extraction process (step a) comprises homogenising the plant in a solvent and liquid-liquid extracting the resin glycoside as a crude extract. As a general example, the plant can be homogenized by a mixer and the compound extracted. The extract can be filtered and concentrated. The extract can be further extracted another time for obtaining a crude extract.

In some embodiments, the homogenisation is performed in ethanol. In other embodiments, the homogenisation is performed in dichloromethane (DCM). The homogenised plant can be further macerated to improve the extraction process. In some embodiments, the liquid-liquid extraction comprises extracting with ethyl acetate. In other embodiments, the liquid-liquid extraction comprises extracting with methanol. In other embodiments, the liquid-liquid extraction comprises extracting using a mixture of hexane: methanol: water in the ratio of 1:1:0.05 (v/v/v).

As will be shown herein, the crude extract obtainable after this step has a lipase inhibition activity IC₅₀ value of 33 µg/mL. It was found that this extract comprises a mixture of at least 3 resin glycosides. The resin glycosides can be selected from the group consisting of Batataoside I, Batataoside II, Batatoside III, Pescaprein XXVII, Batatoside H, Batatoside F, resin glycoside of Formula (I), resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII).

In some embodiments, when extracted from 1 kg of sweet potato (tuber or leaves), or Kangkong, a crude extract of about 0.1%w/w to about 0.3%w/w (relative to the raw material mass) is obtainable. For example, an ethanol extract of about 0.1% to about 0.3% w/w (relative to the raw material mass) is obtainable, and a dichloromethane extract of about 0.19%w/w to about 0.27%w/w (relative to the raw material mass) is obtainable. As is mentioned herein, the extract can contains about 2% to about 30% resin glycosides. In this regard, in some embodiments, the extract comprises at least 2% of resin glycosides, or at least 3%, 4%, 5%, 6%, 7%, 8%, 9%, 10%, 15%, 20%, 25%, or 30% of resin glycosides. In some embodiments, at least 20 mg of resin glycoside is extractable when extracted from 1 kg of the plant. In other embodiments, at least 30 mg is extractable, at least 40 mg is extractable, at least 50 mg is extractable, at least 60 mg is extractable, at least 70 mg is extractable, at least 80 mg is extractable, at least 90 mg is extractable, at least 100 mg is extractable, at least 150 mg is extractable, at least 200 mg is extractable, at least 250 mg is extractable, at least 300 mg is extractable, at least 350 mg is extractable, or at least 400 mg is extractable, or at least 500 mg is extractable, or at least 700 mg is extractable, or at least 1 g is extractable.

The method comprises a first purification step of purifying the crude extract for obtaining a partially purified extract. In some embodiments, the step of purifying the crude extract (step b) comprises fractionating the crude extract. For example, the crude extract can be purified by silica gel column chromatography. Fractions can be collected and the pancreatic lipid (PL) inhibition activity tested for each fraction.

In some embodiments, the step of purifying the crude extract (step b) comprises eluting the crude extract through a liquid chromatography column with a predetermined mobile phase. In some embodiments, the mobile phase is a gradient of hexane:ethyl acetate (hexane:EtOAc; 1:1) to pure ethyl acetate, followed by pure methanol. In other embodiments, the mobile phase is a gradient of dichloromethane:methanol (DCM:MeOH; 7:1) to pure MeOH. In other embodiments, the mobile phase is a gradient of H₂O:MeOH (100:0) to pure MeOH.

Depending on the analysis, another round of silica gel column chromatography can be performed to further purify the collected fractions. Several rounds of chromatographic separation can be performed to improve the purity.

At this step, the extract obtainable has a lipase inhibition activity IC₅₀ value of about 15 µg/mL to about 30 µg/mL. It was found that this extract comprises a mixture of at least 2 resin glycosides. The resin glycosides can be selected from the group consisting of Batataoside I, Batataoside II, Batatoside III, Pescaprein XXVII, Batatoside H, Batatoside F, resin glycoside of Formula (I), resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII).

The method comprises a second purification step by further purifying the partially purified extract for obtaining the resin glycoside. In some embodiments, the further purifying step (step c) comprises fractionating the partially purified extract. The resin glycoside can be further purified by passing it through a high pressure liquid chromatography (HPLC) column and collecting an appropriate fraction. In some embodiments, the purifying step is performed using a reversed-phase C18 column. The skilled person would understand that other methods, such as a semi-preparative HPLC method would also work.

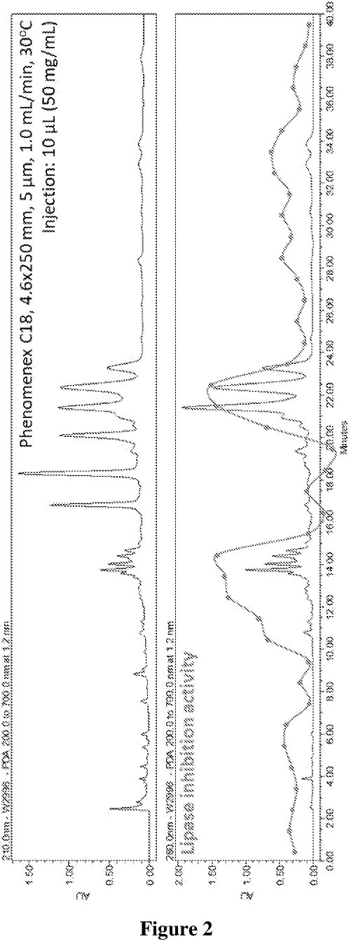

In other embodiments, the further purifying step (step c) comprises eluting the partially purified extract through a liquid chromatography column. In some embodiments, the further purifying step (step c) comprises eluting the partially purified extract through a liquid chromatography column with a predetermined mobile phase. In some embodiments, the further purifying step (step c) comprises eluting the partially purified extract through a liquid chromatography column with a mobile phase having a gradient of MeOH—H₂O (90:10) to MeOH-H₂O (100:0). It was found this this step is advantageous for removing most of the impurities in the partially purified extract (FIG. 2).

The partially purified extract can be subjected to a further purification using a different elution method. For example, further purification can be performed using isocratic elution with pure CH₃CN. In some embodiments, the partially purified extract is further eluted using acetonitrile. In some embodiments, the further purifying step (step c) further comprises eluting a faction from the liquid chromatography column with a mobile phase having a gradient of MeOH—H₂O (90:10) to MeOH—H₂O (100:0) with acetonitrile. Alternatively, the faction from the liquid chromatography column with a mobile phase having a gradient of MeOH-H₂O (90:10) to MeOH—H₂O (100:0) can be further eluted using acetonitrile (ACN)—MeOH (80:20). This was found to be advantageous for separating and resolving the different resin glycosides as well as to remove further impurities from the partially purified extract.

Using the method as disclosed herein, it was found that the purity of resin glycoside can be greatly improved. It was found that this extract comprises at least 1 resin glycoside. The resin glycoside can be selected from the group consisting of Batataoside I, Batataoside II, Batatoside III, Pescaprein XXVII, Batatoside F, Batatoside H, resin glycoside of Formula (I), resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII). Advantageously, the inventors have also found that the IC₅₀ value can be improved. For example, the crude extract has a lipase inhibition with IC₅₀ of 33 µg/mL. After the purification steps were performed, a lipase inhibition with IC₅₀ of about 6 µg/mL can be obtained. Without wanting to be bound by theory, the inventors believe that this is due to the hindrance of binding to lipids by the oxidised/impure (inactive) forms of resin glycosides as well as from the impurities. By efficiently removing these oxidised forms, resin glycosides can more efficiently target the lipids and effect its intended weight loss function. It is also because of these impurities that the weight loss effect from, for example, eating sweet potato or kangkong alone, is minimal. In this regard, by extracting an edible form of resin glycoside from these plants, a better effect can be obtained.

Accordingly, in some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato) and Ipomoea aquatica (kangkong);
wherein the resin glycoside is selected from the group consisting of Batataoside I, Batataoside II, Batatoside III, Pescaprein XXVII, Batatoside F, Batatoside H, a resin glycoside of Formula (I):

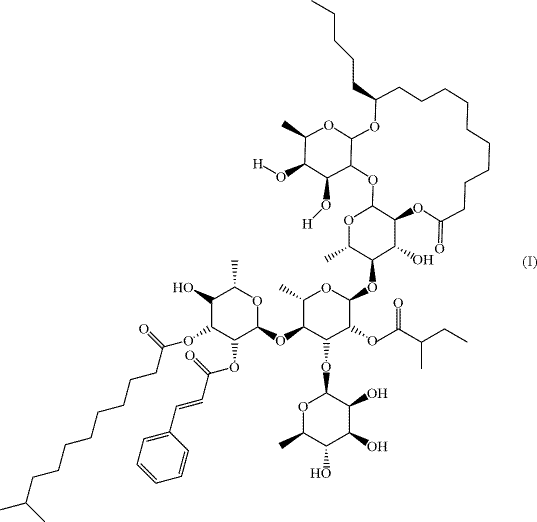

a resin glycoside of Formula (II):

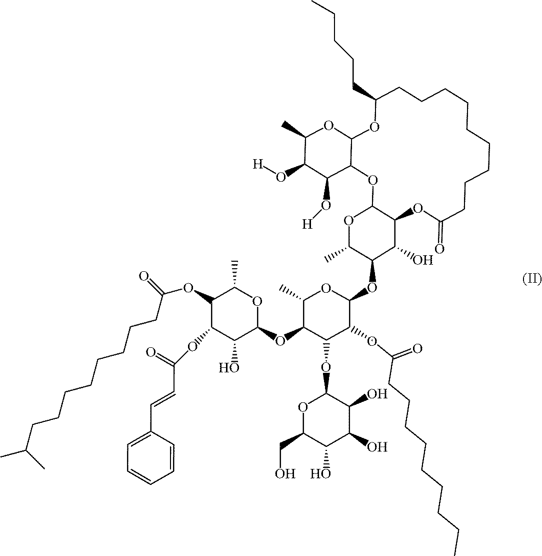

Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII).

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato);
wherein the resin glycoside is selected from the group consisting of Batataoside I, Batataoside II, Batatoside III, Pescaprein XXVII, Batatoside F, Batatoside H, a resin glycoside of Formula (I):

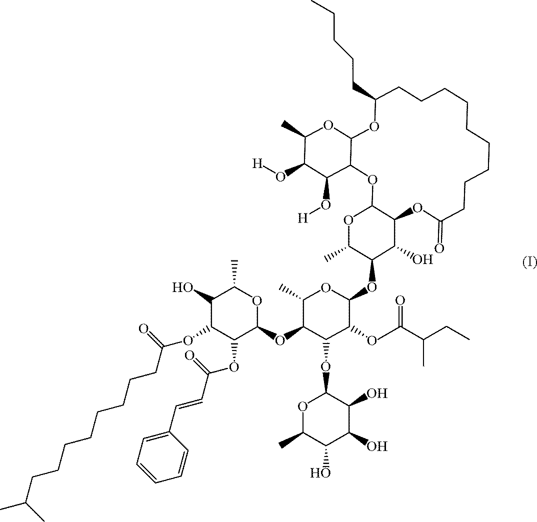

a resin glycoside of Formula (II):

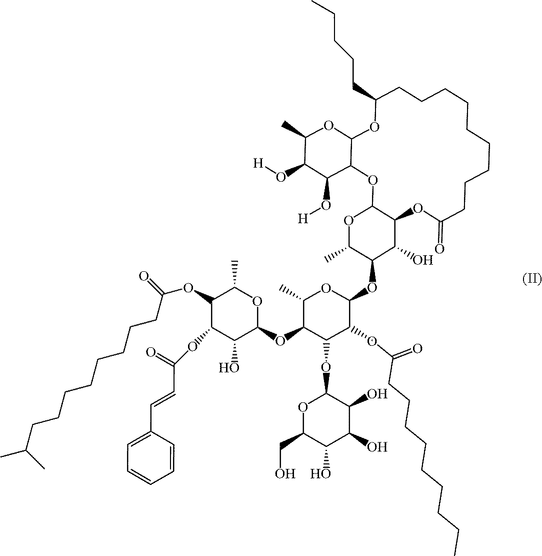

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato) and/or Ipomoea aquatica (kangkong);
wherein the resin glycoside is selected from the group consisting of Batataoside I, Batataoside II, Batatoside III, Pescaprein XXVII, Batatoside F and Batatoside H and a combination thereof; and
wherein the further purifying step (step c) comprises eluting the partially purified extract through a liquid chromatography column with a mobile phase having a gradient of MeOH—H₂O (90:10) to MeOH—H₂O (100:0).

Accordingly, in some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato) and Ipomoea aquatica (kangkong);
wherein the resin glycoside is selected from the group consisting of Batataoside I, Batataoside II, Batatoside III, Pescaprein XXVII, Batatoside F, Batatoside H, a resin glycoside of Formula (I), a resin glycoside of Formula (II), or a combination thereof; and
wherein the further purifying step (step c) comprises eluting the partially purified extract through a liquid chromatography column with a mobile phase having a gradient of MeOH—H₂O (90:10) to MeOH—H₂O (100:0).

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato) and Ipomoea aquatica (kangkong;
wherein the resin glycoside is selected from the group consisting of Batataoside I, Batataoside II, Batatoside III, Pescaprein XXVII, Batatoside F and Batatoside H and a combination thereof; and
wherein the further purifying step (step c) comprises eluting the partially purified extract through a liquid chromatography column with a mobile phase having a gradient of MeOH—H₂O (90:10) to MeOH—H₂O (100:0) and eluting a faction from the liquid chromatography column with a mobile phase having a gradient of MeOH—H₂O (90:10) to MeOH—H₂O (100:0) with acetonitrile (ACN) or with ACN—MeOH (80:20).

Accordingly, in some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea batatas (sweet potato) and Ipomoea aquatica (kangkong);
wherein the resin glycoside is selected from the group consisting of Batataoside I, Batataoside II, Batatoside III, Pescaprein XXVII, Batatoside F, Batatoside H, a resin glycoside of Formula (I), a resin glycoside of Formula (II), or a combination thereof; and
wherein the further purifying step (step c) comprises eluting the partially purified extract through a liquid chromatography column with a mobile phase having a gradient of MeOH—H₂O (90:10) to MeOH—H₂O (100:0) (ACN) or with ACN—MeOH (80:20).

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea aquatica (kangkong);
wherein the resin glycoside is selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII).

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea aquatica (kangkong);
wherein the resin glycoside is selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII); and
wherein the further purifying step (step c) comprises eluting the partially purified extract through a liquid chromatography column with a mobile phase having a gradient of MeOH—H₂O (90:10) to MeOH—H₂O (100:0).

Accordingly, in some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea aquatica (kangkong);
wherein the resin glycoside is selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII); and
wherein the further purifying step (step c) comprises eluting the partially purified extract through a liquid chromatography column with a mobile phase having a gradient of MeOH—H₂O (90:10) to MeOH—H₂O (100:0).

In some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea aquatica (kangkong;
wherein the resin glycoside is selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII); and
wherein the further purifying step (step c) comprises eluting the partially purified extract through a liquid chromatography column with a mobile phase having a gradient of MeOH—H₂O (90:10) to MeOH—H₂O (100:0) and eluting a faction from the liquid chromatography column with a mobile phase having a gradient of MeOH—H₂O (90:10) to MeOH—H₂O (100:0) with acetonitrile (ACN) or with ACN—MeOH (80:20).

Accordingly, in some embodiments, the method of extracting at least one resin glycoside from a plant selected from the Convolvulaceae family, comprises:
a) subjecting the plant to a solvent extraction process for obtaining a crude extract;
b) purifying the crude extract for obtaining a partially purified extract; and
c) further purifying the partially purified extract for obtaining the resin glycoside;
wherein the plant is selected from Ipomoea aquatica (kangkong);
wherein the resin glycoside is selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII); and
wherein the further purifying step (step c) comprises eluting the partially purified extract through a liquid chromatography column with a mobile phase having a gradient of MeOH—H₂O (90:10) to MeOH—H₂O (100:0) (ACN) or with ACN—MeOH (80:20).

The present invention also discloses an extract, comprising:
at least one resin glycoside selected from the group consisting of Batataoside I, Batataoside II, Batatoside III, Pescaprein XXVII, Batatoside F, Batatoside H, resin glycoside of Formula (I), resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea batatas (sweet potato) and/or Ipomoea aquatica (kangkong).

In some embodiments, the extract comprises:

at least one resin glycoside selected from the group consisting of Batataoside I, Batataoside II, Batatoside III, Pescaprein XXVII, Batatoside F and Batatoside H, a resin glycoside of Formula (I), a resin glycoside of Formula (II), or a combination thereof.

In some embodiments, the extract comprises:
at least one resin glycoside selected from the group consisting of Batataoside I, Batataoside II, Batatoside III, Pescaprein XXVII, Batatoside F and Batatoside H, a resin glycoside of Formula (I), a resin glycoside of Formula (II) or a combination thereof;
wherein the resin glycoside is extracted from a plant selected from Ipomoea batatas (sweet potato) and/or Ipomoea aquatica (kangkong).

In some embodiments, the extract comprises:
at least one resin glycoside selected from the group consisting of Batataoside I, Batataoside II, Batatoside III, Pescaprein XXVII, Batatoside F and Batatoside H, a resin glycoside of Formula (I), a resin glycoside of Formula (II) or a combination thereof;
wherein the resin glycoside is extracted from a plant selected from Ipomoea batatas (sweet potato).

In some embodiments, the extract comprising at least one resin glycoside selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);

wherein the resin glycoside is extracted from a plant selected from Ipomoea aquatica (kangkong).

In this regard, the extract can have a lipase inhibition activity IC₅₀ value of less than 15 µg/mL. In other embodiments, the extract has an IC₅₀ value of less than 14 µg/mL, 13 µg/mL, 12 µg/mL, 11 µg/mL, 10 µg/mL, 9 µg/mL, 8 µg/mL, 7 µg/mL, or 6 µg/mL. In other embodiments, the extract has a lipase inhibition activity IC₅₀ value of about 4 µg/mL to about 15 µg/mL.

In some embodiments, the extract comprises resin glycoside which is at least 80% pure; i.e. the extract comprises at least 80% of resin glycoside by weight. In other embodiments, the resin glycoside is at least 85% pure, at least 90% pure, at least 92% pure, at least 94% pure, at least 96% pure, at least 98% pure or at least 99% pure.

In other embodiments, the extract (or partially purified extract) comprises:
at least two resin glycosides selected from the group consisting of Batataoside I, Batataoside II, Batatoside III, Pescaprein XXVII, Batatoside F, Batatoside H, a resin glycoside of Formula (I), a resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea batatas (sweet potato) and/or Ipomoea aquatica (kangkong).

In other embodiments, the extract (or partially purified extract) comprises:
at least two resin glycosides selected from the group consisting of Batataoside I, Batataoside II, Batatoside III, Pescaprein XXVII, Batatoside F, Batatoside H, a resin glycoside of Formula (I), a resin glycoside of Formula (II) or a combination thereof;
wherein the resin glycoside is extracted from a plant selected from Ipomoea batatas (sweet potato).

In other embodiments, the extract (or partially purified extract) comprises:
at least two resin glycosides selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea aquatica (kangkong).

In some embodiments, the extract (or partially purified extract) can be characterised by the impurities present. For example, impurities in sweet potato tuber can be mixture of oligosaccharides and polysaccharides, while in leave extract are mainly chlorophyll and polyphenols, vitamin E, carotenoids, lipids, and fatty acids. The amount of impurities depends on the degree of purification.

In some embodiments, when the extract is at least 80% pure, oligosaccharides and polysaccharides is present at about 15% to about 20% in the extract. In other embodiments, when the extract is at least 80% pure, chlorophyll and polyphenols is present at about 1% to about 20% in the extract, about 1% to about 18%, about 1% to about 16%, about 1% to about 15%, about 1% to about 14%, about 1% to about 12%, about 1% to about 10%, about 1% to about 8%, about 1% to about 6%, about 1% to about 5%, about 1% to about 4%, about 1% to about 3%, or about 1% to about 2%. In other embodiments, when the extract is at least 80% pure, vitamin E is present at about 1% to about 20% in the extract, about 1% to about 18%, about 1% to about 16%, about 1% to about 15%, about 1% to about 14%, about 1% to about 12%, about 1% to about 10%, about 1% to about 8%, about 1% to about 6%, about 1% to about 5%, about 1% to about 4%, about 1% to about 3%, or about 1% to about 2%. In other embodiments, when the extract is at least 80% pure, carotenoids is present at about 1% to about 20% in the extract, about 1% to about 18%, about 1% to about 16%, about 1% to about 15%, about 1% to about 14%, about 1% to about 12%, about 1% to about 10%, about 1% to about 8%, about 1% to about 6%, about 1% to about 5%, about 1% to about 4%, about 1% to about 3%, or about 1% to about 2%. In other embodiments, when the extract is at least 80% pure, lipids and fatty acids is present at about 1% to about 20% in the extract, about 1% to about 18%, about 1% to about 16%, about 1% to about 15%, about 1% to about 14%, about 1% to about 12%, about 1% to about 10%, about 1% to about 8%, about 1% to about 6%, about 1% to about 5%, about 1% to about 4%, about 1% to about 3%, or about 1% to about 2%.

In some embodiments, when the extract is at least 90% pure, oligosaccharides and polysaccharides is present at about 5% to about 10% in the extract. In other embodiments, when the extract is at least 90% pure, chlorophyll and polyphenols is present at about 1% to about 10% in the extract, about 1% to about 8%, about 1% to about 6%, about 1% to about 5%, about 1% to about 4%, about 1% to about 3%, or about 1% to about 2%. In other embodiments, when the extract is at least 90% pure, vitamin E is present at about 1% to about 10% in the extract, about 1% to about 8%, about 1% to about 6%, about 1% to about 5%, about 1% to about 4%, about 1% to about 3%, or about 1% to about 2%. In other embodiments, when the extract is at least 90% pure, carotenoids is present at about 1% to about 10% in the extract, about 1% to about 8%, about 1% to about 6%, about 1% to about 5%, about 1% to about 4%, about 1% to about 3%, or about 1% to about 2%. In other embodiments, when the extract is at least 90% pure, lipids and fatty acids is present at about 1% to about 10% in the extract, about 1% to about 8%, about 1% to about 6%, about 1% to about 5%, about 1% to about 4%, about 1% to about 3%, or about 1% to about 2%.

In some embodiments, the extract comprises Batatoside F at about 2 mg/g to about 40 mg/g relative to the extract. In other embodiments, the amount is about 2 mg/g to about 35 mg/g, about 2 mg/g to about 30 mg/g, about 2 mg/g to about 25 mg/g, about 2 mg/g to about 20 mg/g, about 2 mg/g to about 15 mg/g, about 2 mg/g to about 12 mg/g, about 3 mg/g to about 12 mg/g, about 4 mg/g to about 12 mg/g, about 4 mg/g to about 11 mg/g, about 4 mg/g to about 10 mg/g, about 4 mg/g to about 9 mg/g, about 5 mg/g to about 9 mg/g, or about 5 mg/g to about 8 mg/g.

In some embodiments, the extract comprises Batatoside H at about 2 mg/g to about 8 mg/g relative to the extract. In other embodiments, the amount is about 2 mg/g to about 7 mg/g, about 2.5 mg/g to about 7 mg/g, about 3 mg/g to about 7 mg/g, about 3 mg/g to about 6 mg/g, about 3.5 mg/g to about 6 mg/g, or about 3.5 mg/g to about 6.5 mg/g.

In some embodiments, the extract comprises Batataoside I at about 0.5 mg/g to about 500 mg/g relative to the extract. In some embodiments, the amount is about 0.5 mg/g to about 450 mg/g, about 0.5 mg/g to about 400 mg/g, about 0.5 mg/g to about 350 mg/g, about 0.5 mg/g to about 300 mg/g, about 0.5 mg/g to about 250 mg/g, about 0.5 mg/g to about 200 mg/g, about 0.5 mg/g to about 150 mg/g, about 0.5 mg/g to about 100 mg/g, about 0.5 mg/g to about 80 mg/g, about 0.5 mg/g to about 60 mg/g, about 0.5 mg/g to about 40 mg/g, about 0.5 mg/g to about 20 mg/g, about 0.5 mg/g to about 10 mg/g, about 0.5 mg/g to about 8 mg/g, about 0.5 mg/g to about 6 mg/g, about 0.5 mg/g to about 5 mg/g, about 0.5 mg/g to about 3.5 mg/g, about 0.5 mg/g to about 3 mg/g, about 0.5 mg/g to about 2.5 mg/g, about 0.6 mg/g to about 2.5 mg/g, about 0.7 mg/g to about 2.5 mg/g, about 0.8 mg/g to about 2.5 mg/g, about 0.9 mg/g to about 2.5 mg/g, or about 0.9 mg/g to about 2 mg/g.

In some embodiments, the extract comprises Batataoside III at about 0.1 mg/g to about 10 mg/g relative to the extract. In other embodiments, the amount is about 0.1 mg/g to about 8 mg/g, about 0.1 mg/g to about 6 mg/g, about 0.1 mg/g to about 4 mg/g, about 0.1 mg/g to about 3.5 mg/g, about 0.1 mg/g to about 3 mg/g, about 0.1 mg/g to about 2.5 mg/g, about 0.1 mg/g to about 2 mg/g, about 0.1 mg/g to about 1.5 mg/g, about 0.1 mg/g to about 1 mg/g, about 0.2 mg/g to about 1 mg/g, or about 0.4 mg/g to about 1 mg/g.

In some embodiments, the extract comprises Batataoside II at about 0.1 mg/g to about 5 mg/g relative to the extract. In other embodiments, the amount is about 0.1 mg/g to about 4 mg/g, about 0.1 mg/g to about 3.5 mg/g, about 0.1 mg/g to about 3 mg/g, about 0.1 mg/g to about 2.5 mg/g, about 0.1 mg/g to about 2 mg/g, about 0.1 mg/g to about 1.5 mg/g, about 0.1 mg/g to about 1 mg/g, about 0.2 mg/g to about 1 mg/g, about 0.4 mg/g to about 1 mg/g, or about 0.4 mg/g to about 0.9 mg/g.

In some embodiments, the extract comprises Pescaprein XXVII at about 0.1 mg/g to about 5 mg/g relative to the extract. In other embodiments, the amount is about 0.1 mg/g to about 4 mg/g, about 0.1 mg/g to about 3.5 mg/g, about 0.1 mg/g to about 3 mg/g, about 0.1 mg/g to about 2.5 mg/g, about 0.1 mg/g to about 2 mg/g, about 0.1 mg/g to about 1.5 mg/g, about 0.1 mg/g to about 1 mg/g, about 0.2 mg/g to about 1 mg/g, or about 0.4 mg/g to about 1 mg/g.

In some embodiments, the extract comprises resin glycoside of Formula (I) at about 0.1 mg/g to about 4 mg/g relative to the extract. In other embodiments, the amount is about 0.1 mg/g to about 3.5 mg/g, about 0.1 mg/g to about 3 mg/g, about 0.1 mg/g to about 2.5 mg/g, about 0.1 mg/g to about 2 mg/g, about 0.1 mg/g to about 1.5 mg/g, about 0.1 mg/g to about 1 mg/g, about 0.2 mg/g to about 1 mg/g, about 0.4 mg/g to about 1 mg/g, or about 0.4 mg/g to about 0.9 mg/g.

In some embodiments, the extract comprises resin glycoside of Formula (II) at about 10 mg/g to about 500 mg/g relative to the extract. In some embodiments, the amount is about 10 mg/g to about 450 mg/g, about 10 mg/g to about 400 mg/g, about 10 mg/g to about 350 mg/g, about 10 mg/g to about 300 mg/g, about 10 mg/g to about 250 mg/g, about 10 mg/g to about 200 mg/g, about 10 mg/g to about 150 mg/g, about 10 mg/g to about 100 mg/g, about 10 mg/g to about 80 mg/g, about 10 mg/g to about 60 mg/g, about 10 mg/g to about 40 mg/g, about 10 mg/g to about 20 mg/g, or about 10 mg/g to about 15 mg/g.

In some embodiments, the extract comprises a mixture of at least 2 resin glycosides. In other embodiments, the extract comprises a mixture of at least 3 resin glycosides. For example, the extract can comprise resin glycosides in the following mixtures:

| | | | | | | | |
|---|---|---|---|---|---|---|---|
| Batataoside I | Batataoside II | Batataoside III | Batataoside F | Batataoside H | Pescaprein XXVII | Formula (1) | Formula (II) |
| present | present | | | | | | |
| present | | present | | | | | |
| present | | | present | | | | |
| present | | | | present | | | |
| present | | | | | present | | |
| present | | | | | | present | |
| present | | | | | | | present |
| | present | present | | | | | |
| | present | | present | | | | |
| | present | | | present | | | |
| | present | | | | present | | |
| | present | | | | | present | |
| | present | | | | | | present |
| | | present | present | | | | |
| | | present | | present | | | |
| | | present | | | present | | |
| | | present | | | | present | |
| | | present | | | | | present |
| | | | present | present | | | |
| | | | present | | present | | |
| | | | present | | | present | |
| | | | present | | | | present |
| | | | | present | present | | |
| | | | | present | | present | |
| | | | | present | | | present |
| | | | | | present | present | |
| | | | | | present | | present |
| | | | | | | present | present |
| present | present | present | | | | | |
| present | present | | present | | | | |
| present | present | | | present | | | |
| present | present | | | | present | | |
| present | present | | | | | present | |
| present | present | | | | | | present |
| present | | present | present | | | | |
| present | | present | | present | | | |
| present | | present | | | present | | |
| present | | present | | | | present | |
| present | | present | | | | | present |
| present | | | present | present | | | |
| present | | | present | | present | | |
| present | | | present | | | present | |
| present | | | present | | | | present |
| present | | | | present | present | | |
| present | | | | present | | present | |
| present | | | | present | | | present |
| present | | | | | present | present | |
| present | | | | | present | | present |
| present | | | | | | present | present |
| | present | present | present | | | | |
| | present | present | | present | | | |
| | present | present | | | present | | |
| | present | present | | | | present | |
| | present | present | | | | | present |
| | present | | present | present | | | |
| | present | | present | | present | | |
| | present | | present | | | present | |
| | present | | present | | | | present |
| | present | | | present | present | | |
| | present | | | present | | present | |
| | present | | | present | | | present |
| | present | | | | present | present | |
| | present | | | | present | | present |
| | present | | | | | present | present |
| | | present | present | present | | | |
| | | present | present | | present | | |
| | | present | present | | | present | |
| | | present | present | | | | present |
| | | present | | present | present | | |
| | | present | | present | | present | |
| | | present | | present | | | present |
| | | present | | | present | present | |
| | | present | | | present | | present |
| | | present | | | | present | present |
| | | | present | present | present | | |
| | | | present | present | | present | |
| | | | present | present | | | present |
| | | | present | | present | present | |
| | | | present | | present | | present |
| | | | present | | | present | present |
| | | | | present | present | present | |
| | | | | present | present | | present |
| | | | | present | | present | present |
| | | | | | present | present | present |

In some embodiments, the extract comprises Batatoside E at about 0.5 mg/g to about 500 mg/g relative to the extract. In other embodiments, the extract comprises Batatoside D at about 0.5 mg/g to about 500 mg/g relative to the extract. In other embodiments, the extract comprises Aquaterin VI at about 0.5 mg/g to about 500 mg/g relative to the extract. In other embodiments, the extract comprises Aquaterin V at about 0.5 mg/g to about 500 mg/g relative to the extract. In other embodiments, the extract comprises Aquaterin XI at about 0.5 mg/g to about 500 mg/g relative to the extract. In other embodiments, the extract comprises Aquaterin II at about 0.5 mg/g to about 500 mg/g relative to the extract. In other embodiments, the extract comprises Stoloniferin VIII at about 0.5 mg/g to about 500 mg/g relative to the extract. In other embodiments, the extract comprises Aquaterin XIV at about 0.5 mg/g to about 500 mg/g relative to the extract. In other embodiments, the extract comprises Batatoside C at about 0.5 mg/g to about 500 mg/g relative to the extract. In other embodiments, the extract comprises Batatinoside I at about 0.5 mg/g to about 500 mg/g relative to the extract. In other embodiments, the extract comprises Aquaterin VII at about 0.5 mg/g to about 500 mg/g relative to the extract. In other embodiments, the extract comprises Aquaterin III at about 0.5 mg/g to about 500 mg/g relative to the extract. In other embodiments, the extract comprises Murucoidin XVIII at about 0.5 mg/g to about 500 mg/g relative to the extract. In other embodiments, the extract comprises Aquaterin XIII at about 0.5 mg/g to about 500 mg/g relative to the extract. In other embodiments, the extract comprises Aquaterin XII at about 0.5 mg/g to about 500 mg/g relative to the extract. In other embodiments, the extract comprises resin glycoside of Formula (III) at about 0.5 mg/g to about 500 mg/g relative to the extract. In other embodiments, the extract comprises resin glycoside of Formula (IV) at about 0.5 mg/g to about 500 mg/g relative to the extract.

In other embodiments, the extract comprises resin glycoside of Formula (V) at about 0.5 mg/g to about 500 mg/g relative to the extract. In other embodiments, the extract comprises resin glycoside of Formula (VI) at about 0.5 mg/g to about 500 mg/g relative to the extract. In other embodiments, the extract comprises resin glycoside of Formula (VII) at about 0.5 mg/g to about 500 mg/g relative to the extract. In some embodiments, the amount is about 0.5 mg/g to about 450 mg/g, about 0.5 mg/g to about 400 mg/g, about 0.5 mg/g to about 350 mg/g, about 0.5 mg/g to about 300 mg/g, about 0.5 mg/g to about 250 mg/g, about 0.5 mg/g to about 200 mg/g, about 0.5 mg/g to about 150 mg/g, about 0.5 mg/g to about 100 mg/g, about 0.5 mg/g to about 80 mg/g, about 0.5 mg/g to about 60 mg/g, about 0.5 mg/g to about 40 mg/g, about 0.5 mg/g to about 20 mg/g, about 0.5 mg/g to about 10 mg/g, about 0.5 mg/g to about 8 mg/g, about 0.5 mg/g to about 6 mg/g, about 0.5 mg/g to about 5 mg/g, about 0.5 mg/g to about 3.5 mg/g, about 0.5 mg/g to about 3 mg/g, about 0.5 mg/g to about 2.5 mg/g, about 0.6 mg/g to about 2.5 mg/g, about 0.7 mg/g to about 2.5 mg/g, about 0.8 mg/g to about 2.5 mg/g, about 0.9 mg/g to about 2.5 mg/g, or about 0.9 mg/g to about 2 mg/g.

For example, the extract can comprise resin glycosides in the following mixtures:

| | | | | | | | | | | | | | | | | | | | | |
|---|---|---|---|---|---|---|---|---|---|---|---|---|---|---|---|---|---|---|---|---|
| Batatoside F | Batatoside E | Batatoside D | Aquaterin VI | Aquaterin V | Aquaterin XI | Aquaterin II | Stoloniferin | Aquaterin XIV | Batatoside C | Batatinoside I | Aquaterin VII | Aquaterin III | Murucoidin | Aquaterin III | Aqauterin XII | Formula (III) | Formula (IV) | Formula (V) | Formula (VI) | Formula (VII) |
| P | P | | | | | | | | | | | | | | | | | | | |
| P | | P | | | | | | | | | | | | | | | | | | |
| P | | | P | | | | | | | | | | | | | | | | | |
| P | | | | P | | | | | | | | | | | | | | | | |
| P | | | | | P | | | | | | | | | | | | | | | |
| P | | | | | | P | | | | | | | | | | | | | | |
| P | | | | | | | P | | | | | | | | | | | | | |
| P | | | | | | | | P | | | | | | | | | | | | |
| P | | | | | | | | | P | | | | | | | | | | | |
| P | | | | | | | | | | P | | | | | | | | | | |
| P | | | | | | | | | | | P | | | | | | | | | |
| P | | | | | | | | | | | | P | | | | | | | | |
| P | | | | | | | | | | | | | P | | | | | | | |
| P | | | | | | | | | | | | | | P | | | | | | |
| P | | | | | | | | | | | | | | | P | | | | | |
| P | | | | | | | | | | | | | | | | P | | | | |
| P | | | | | | | | | | | | | | | | | P | | | |
| P | | | | | | | | | | | | | | | | | | P | | |
| P | | | | | | | | | | | | | | | | | | | P | |
| P | | | | | | | | | | | | | | | | | | | | P |
| | P | P | | | | | | | | | | | | | | | | | | |
| | P | | P | | | | | | | | | | | | | | | | | |
| | P | | | P | | | | | | | | | | | | | | | | |
| | P | | | | P | | | | | | | | | | | | | | | |
| | P | | | | | P | | | | | | | | | | | | | | |
| | P | | | | | | P | | | | | | | | | | | | | |
| | P | | | | | | | P | | | | | | | | | | | | |
| | P | | | | | | | | P | | | | | | | | | | | |
| | P | | | | | | | | | P | | | | | | | | | | |
| | P | | | | | | | | | | P | | | | | | | | | |
| | P | | | | | | | | | | | P | | | | | | | | |
| | P | | | | | | | | | | | | P | | | | | | | |
| | P | | | | | | | | | | | | | P | | | | | | |
| | P | | | | | | | | | | | | | | P | | | | | |
| | P | | | | | | | | | | | | | | | P | | | | |
| | P | | | | | | | | | | | | | | | | P | | | |
| | P | | | | | | | | | | | | | | | | | P | | |
| | P | | | | | | | | | | | | | | | | | | P | |
| | P | | | | | | | | | | | | | | | | | | | P |
| | | P | P | | | | | | | | | | | | | | | | | |
| | | P | | P | | | | | | | | | | | | | | | | |
| | | P | | | P | | | | | | | | | | | | | | | |
| | | P | | | | P | | | | | | | | | | | | | | |
| | | P | | | | | P | | | | | | | | | | | | | |
| | | P | | | | | | P | | | | | | | | | | | | |
| | | P | | | | | | | P | | | | | | | | | | | |
| | | P | | | | | | | | P | | | | | | | | | | |
| | | P | | | | | | | | | P | | | | | | | | | |
| | | P | | | | | | | | | | P | | | | | | | | |
| | | P | | | | | | | | | | | P | | | | | | | |
| | | P | | | | | | | | | | | | P | | | | | | |
| | | P | | | | | | | | | | | | | P | | | | | |
| | | P | | | | | | | | | | | | | | P | | | | |
| | | P | | | | | | | | | | | | | | | P | | | |
| | | P | | | | | | | | | | | | | | | | P | | |
| | | P | | | | | | | | | | | | | | | | | P | |
| | | P | | | | | | | | | | | | | | | | | | P |
| | | | P | P | | | | | | | | | | | | | | | | |
| | | | P | | P | | | | | | | | | | | | | | | |
| | | | P | | | P | | | | | | | | | | | | | | |
| | | | P | | | | P | | | | | | | | | | | | | |
| | | | P | | | | | P | | | | | | | | | | | | |
| | | | P | | | | | | P | | | | | | | | | | | |
| | | | P | | | | | | | P | | | | | | | | | | |
| | | | P | | | | | | | | P | | | | | | | | | |
| | | | P | | | | | | | | | P | | | | | | | | |
| | | | P | | | | | | | | | | P | | | | | | | |
| | | | P | | | | | | | | | | | P | | | | | | |
| | | | P | | | | | | | | | | | | P | | | | | |
| | | | P | | | | | | | | | | | | | P | | | | |
| | | | P | | | | | | | | | | | | | | P | | | |
| | | | P | | | | | | | | | | | | | | | P | | |
| | | | P | | | | | | | | | | | | | | | | P | |
| | | | P | | | | | | | | | | | | | | | | | P |
| | | | | P | P | | | | | | | | | | | | | | | |
| | | | | P | | P | | | | | | | | | | | | | | |
| | | | | P | | | P | | | | | | | | | | | | | |
| | | | | P | | | | P | | | | | | | | | | | | |
| | | | | P | | | | | P | | | | | | | | | | | |
| | | | | P | | | | | | P | | | | | | | | | | |
| | | | | P | | | | | | | P | | | | | | | | | |
| | | | | P | | | | | | | | P | | | | | | | | |
| | | | | P | | | | | | | | | P | | | | | | | |
| | | | | P | | | | | | | | | | P | | | | | | |
| | | | | P | | | | | | | | | | | P | | | | | |
| | | | | P | | | | | | | | | | | | P | | | | |
| | | | | P | | | | | | | | | | | | | P | | | |
| | | | | P | | | | | | | | | | | | | | P | | |
| | | | | P | | | | | | | | | | | | | | | P | |
| | | | | P | | | | | | | | | | | | | | | | P |
| | | | | | P | P | | | | | | | | | | | | | | |
| | | | | | P | | P | | | | | | | | | | | | | |
| | | | | | P | | | P | | | | | | | | | | | | |
| | | | | | P | | | | P | | | | | | | | | | | |
| | | | | | P | | | | | P | | | | | | | | | | |
| | | | | | P | | | | | | P | | | | | | | | | |
| | | | | | P | | | | | | | P | | | | | | | | |
| | | | | | P | | | | | | | | P | | | | | | | |
| | | | | | P | | | | | | | | | P | | | | | | |
| | | | | | P | | | | | | | | | | P | | | | | |
| | | | | | P | | | | | | | | | | | P | | | | |
| | | | | | P | | | | | | | | | | | | P | | | |
| | | | | | P | | | | | | | | | | | | | P | | |
| | | | | | P | | | | | | | | | | | | | | P | |
| | | | | | P | | | | | | | | | | | | | | | P |
| | | | | | | P | P | | | | | | | | | | | | | |
| | | | | | | P | | P | | | | | | | | | | | | |
| | | | | | | P | | | P | | | | | | | | | | | |
| | | | | | | P | | | | P | | | | | | | | | | |
| | | | | | | P | | | | | P | | | | | | | | | |
| | | | | | | P | | | | | | P | | | | | | | | |
| | | | | | | P | | | | | | | P | | | | | | | |
| | | | | | | P | | | | | | | | P | | | | | | |
| | | | | | | P | | | | | | | | | P | | | | | |
| | | | | | | P | | | | | | | | | | P | | | | |
| | | | | | | P | | | | | | | | | | | P | | | |
| | | | | | | P | | | | | | | | | | | | P | | |
| | | | | | | P | | | | | | | | | | | | | P | |
| | | | | | | P | | | | | | | | | | | | | | P |
| | | | | | | | P | P | | | | | | | | | | | | |
| | | | | | | | P | | P | | | | | | | | | | | |
| | | | | | | | P | | | P | | | | | | | | | | |
| | | | | | | | P | | | | P | | | | | | | | | |
| | | | | | | | P | | | | | P | | | | | | | | |
| | | | | | | | P | | | | | | P | | | | | | | |
| | | | | | | | P | | | | | | | P | | | | | | |
| | | | | | | | P | | | | | | | | P | | | | | |
| | | | | | | | P | | | | | | | | | P | | | | |
| | | | | | | | P | | | | | | | | | | P | | | |
| | | | | | | | P | | | | | | | | | | | P | | |
| | | | | | | | P | | | | | | | | | | | | P | |
| | | | | | | | P | | | | | | | | | | | | | P |
| | | | | | | | | P | P | | | | | | | | | | | |
| | | | | | | | | P | | P | | | | | | | | | | |
| | | | | | | | | P | | | P | | | | | | | | | |
| | | | | | | | | P | | | | P | | | | | | | | |
| | | | | | | | | P | | | | | P | | | | | | | |
| | | | | | | | | P | | | | | | P | | | | | | |
| | | | | | | | | P | | | | | | | P | | | | | |
| | | | | | | | | P | | | | | | | | P | | | | |
| | | | | | | | | P | | | | | | | | | P | | | |
| | | | | | | | | P | | | | | | | | | | P | | |
| | | | | | | | | P | | | | | | | | | | | P | |
| | | | | | | | | P | | | | | | | | | | | | P |
| | | | | | | | | | P | P | | | | | | | | | | |
| | | | | | | | | | P | | P | | | | | | | | | |
| | | | | | | | | | P | | | P | | | | | | | | |
| | | | | | | | | | P | | | | P | | | | | | | |
| | | | | | | | | | P | | | | | P | | | | | | |
| | | | | | | | | | P | | | | | | P | | | | | |
| | | | | | | | | | P | | | | | | | P | | | | |
| | | | | | | | | | P | | | | | | | | P | | | |
| | | | | | | | | | P | | | | | | | | | P | | |
| | | | | | | | | | P | | | | | | | | | | P | |
| | | | | | | | | | P | | | | | | | | | | | P |
| | | | | | | | | | | P | P | | | | | | | | | |
| | | | | | | | | | | P | | P | | | | | | | | |
| | | | | | | | | | | P | | | P | | | | | | | |
| | | | | | | | | | | P | | | | P | | | | | | |
| | | | | | | | | | | P | | | | | P | | | | | |
| | | | | | | | | | | P | | | | | | P | | | | |
| | | | | | | | | | | P | | | | | | | P | | | |
| | | | | | | | | | | P | | | | | | | | P | | |
| | | | | | | | | | | P | | | | | | | | | P | |
| | | | | | | | | | | P | | | | | | | | | | P |
| | | | | | | | | | | | P | P | | | | | | | | |
| | | | | | | | | | | | P | | P | | | | | | | |
| | | | | | | | | | | | P | | | P | | | | | | |
| | | | | | | | | | | | P | | | | P | | | | | |
| | | | | | | | | | | | P | | | | | P | | | | |
| | | | | | | | | | | | P | | | | | | P | | | |
| | | | | | | | | | | | P | | | | | | | P | | |
| | | | | | | | | | | | P | | | | | | | | P | |
| | | | | | | | | | | | P | | | | | | | | | P |
| | | | | | | | | | | | | P | P | | | | | | | |
| | | | | | | | | | | | | P | | P | | | | | | |
| | | | | | | | | | | | | P | | | P | | | | | |
| | | | | | | | | | | | | P | | | | P | | | | |
| | | | | | | | | | | | | P | | | | | P | | | |
| | | | | | | | | | | | | P | | | | | | P | | |
| | | | | | | | | | | | | P | | | | | | | P | |
| | | | | | | | | | | | | P | | | | | | | | P |
| | | | | | | | | | | | | | P | P | | | | | | |
| | | | | | | | | | | | | | P | | P | | | | | |
| | | | | | | | | | | | | | P | | | P | | | | |
| | | | | | | | | | | | | | P | | | | P | | | |
| | | | | | | | | | | | | | P | | | | | P | | |
| | | | | | | | | | | | | | P | | | | | | P | |
| | | | | | | | | | | | | | P | | | | | | | P |
| | | | | | | | | | | | | | | P | P | | | | | |
| | | | | | | | | | | | | | | P | | P | | | | |
| | | | | | | | | | | | | | | P | | | P | | | |
| | | | | | | | | | | | | | | P | | | | P | | |
| | | | | | | | | | | | | | | P | | | | | P | |
| | | | | | | | | | | | | | | P | | | | | | P |
| | | | | | | | | | | | | | | | P | P | | | | |
| | | | | | | | | | | | | | | | P | | P | | | |
| | | | | | | | | | | | | | | | P | | | P | | |
| | | | | | | | | | | | | | | | P | | | | P | |
| | | | | | | | | | | | | | | | P | | | | | P |
| | | | | | | | | | | | | | | | | P | P | | | |
| | | | | | | | | | | | | | | | | P | | P | | |
| | | | | | | | | | | | | | | | | P | | | P | |
| | | | | | | | | | | | | | | | | P | | | | P |
| | | | | | | | | | | | | | | | | | P | P | | |
| | | | | | | | | | | | | | | | | | P | | P | |
| | | | | | | | | | | | | | | | | | P | | | P |
| | | | | | | | | | | | | | | | | | | P | P | |
| | | | | | | | | | | | | | | | | | | P | | P |
| | | | | | | | | | | | | | | | | | | | P | P |

P: present

In this regard, the extract (or partially purified extract) can have a lipase inhibition activity IC₅₀ value of about 15 µg/mL to about 30 µg/mL of extract. In other embodiments, the extract has an IC₅₀ value of about 15 µg/mL to about 27 µg/mL, about 15 µg/mL to about 25 µg/mL, about 15 µg/mL to about 22 µg/mL, or about 15 µg/mL to about 20 µg/mL.

In some embodiments, the extract (or partially purified extract) comprises resin glycoside which is about 50% to about 80% pure. In other embodiments, the resin glycoside is about 50% to about 75% pure, about 50% to about 70% pure, about 50% to about 65% pure, or about 50% to about 60% pure, In other embodiments, the resin glycoside is about 50% pure, about 55% pure, about 60% pure, about 65% pure, about 70% pure, about 75% pure, or about 80% pure.

In other embodiments, the extract (or crude extract) comprises:
at least three resin glycosides selected from the group consisting of Batataoside I, Batataoside II, Batatoside III, Pescaprein XXVII, Batatoside F, Batatoside H, a resin glycoside of Formula (I), a resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea batatas (sweet potato) and/or Ipomoea aquatica (kangkong).

In other embodiments, the extract (or crude extract) comprises:
at least three resin glycosides selected from the group consisting of Batataoside I, Batataoside II, Batatoside III, Pescaprein XXVII, Batatoside F, Batatoside H, a resin glycoside of Formula (I), a resin glycoside of Formula (II) or a combination thereof;
wherein the resin glycoside is extracted from a plant selected from Ipomoea batatas (sweet potato).

In other embodiments, the extract (or crude extract) comprises:
at least three resin glycosides selected from the group consisting of Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII),;
wherein the resin glycoside is extracted from a plant selected from Ipomoea aquatica (kangkong).

Advantageously, resin glycosides with less/shorter fatty acid side chains bonded to the sugar backbone have a higher inhibition effect.

In this regard, the extract (or crude extract) can have a lipase inhibition activity IC₅₀ value of more than 30 µg/mL. In other embodiments, the extract has an IC₅₀ value of more than 31 µg/mL, 32 µg/mL, 33 µg/mL, 35 µg/mL, 40 µg/mL, or 45 µg/mL.

In some embodiments, the extract (or crude extract) comprises resin glycoside which is not less than 30% pure. In other embodiments, the resin glycoside is not less than 35% pure, not less than 40% pure, or not less than 45% pure. In other embodiments, the resin glycoside is about 30% pure, about 35% pure, about 40% pure, about 45% pure, or about 50% pure.

In some embodiments, the extract comprises:
a) Batatoside F at about 2 mg/g to about 12 mg/g; and
b) Batatoside H at about 2 mg/g to about 8 mg/g;
wherein the resin glycoside is extracted from a plant selected from Ipomoea batatas (sweet potato) and/or Ipomoea aquatica (kangkong).

In some embodiments, the extract comprises:
a) Batatoside F at about 2 mg/g to about 12 mg/g;
b) Batatoside H at about 2 mg/g to about 8 mg/g; and
c) Batataoside I at about 0.5 mg/g to about 5 mg/g;
wherein the resin glycoside is extracted from a plant selected from Ipomoea batatas (sweet potato) and/or Ipomoea aquatica (kangkong).

In some embodiments, the extract comprises:
a) Batataoside I at about 0.5 mg/g to about 5 mg/g; and
b) Batataoside II at about 0.1 mg/g to about 5 mg/g;
wherein the resin glycoside is extracted from a plant selected from Ipomoea batatas (sweet potato) and/or Ipomoea aquatica (kangkong).

In some embodiments, the extract comprises:
a) Batataoside I at about 0.5 mg/g to about 5 mg/g;
b) Batataoside II at about 0.1 mg/g to about 5 mg/g; and
c) Batataoside III at about 0.1 mg/g to about 5 mg/g;
wherein the resin glycoside is extracted from a plant selected from Ipomoea batatas (sweet potato) and/or Ipomoea aquatica (kangkong).

The present invention also provides a resin glycoside of Formula (I),

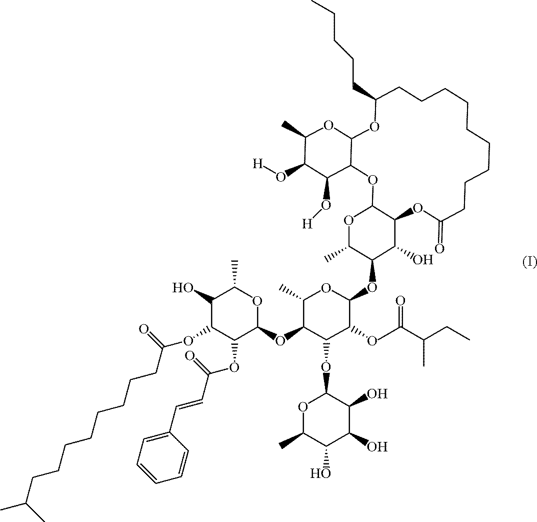

Resin glycoside of Formula (I) can, for example, be extracted from sweet potato tuber.

The present invention also provides a method of extracting resin glycoside of Formula (I) from Ipomoea batatas (sweet potato).

The present invention also provides a resin glycoside of Formula (II),

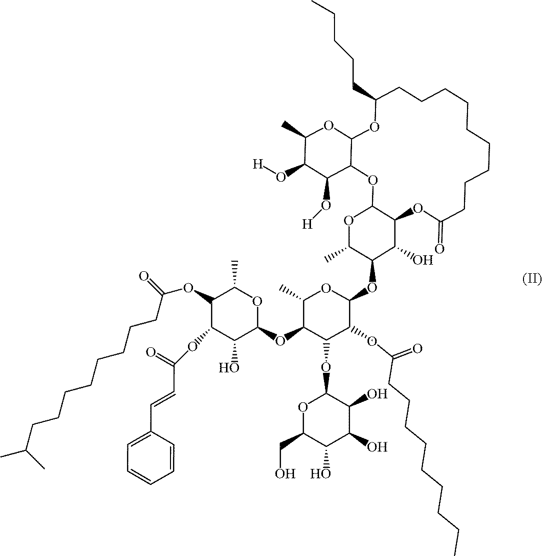

Resin glycoside of Formula (II) can, for example, be extracted from sweet potato leaves.

The present invention also provides a method of extracting resin glycoside of Formula (II) from Ipomoea batatas (sweet potato).

The present invention also provides a Type A resin glycoside of Formula (III), Formula (IV), Formula (V), Formula (VI), and/or Formula (VII),

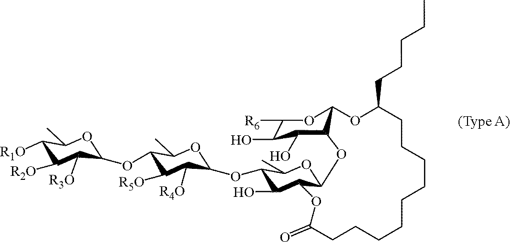

Formula (III) is when R₁ is n-dodecanoyl, R₂ is cinnamoyl, R₃ is 2-methylbutanoyl, R₄ is H, R₅ is rhamnopyranosyl, and R₆ is methyl.

Formula (IV) is when R₁ is isobutanoyl, R₂ is H, R₃ is H, R₄ is n-octanoyl, R₅ is glucopyranosyl, and R₆ is methyl.

Formula (V) is when R₁ is n-octanoyl, R₂ is n-octanoyl, R₃ is H, R₄ is 2-methylbutanoyl, R₅ is n-octanoyl, and R₆ is methyl.

Formula (VI) is when R₁ is n-dodecanoyl, R₂ is H, R₃ is H, R₄ is H, R₅ is n-octanoyl, and R₆ is methyl.

Formula (VII) is when R₁ is n-octanoyl, R₂ is H, R₃ is n-octanoyl, R₄ is 2-methylbutanoyl, R₅ is glucopyranosyl, and R₆ is methyl.

Resin glycoside of Formula (II) can, for example, be extracted from Kangkong.

It is expected that as the extract is from a natural product, there are minimal side effects. This is advantageous compared to Orlistat. The positive effect is similar to Orlistat, while side effects are expected to be milder than Orlistat since the extract is from an edible plant with over 500 years of consumption history. Additionally, the extract has light yellow colour and mild sweetness, which can be advantageous for consumer acceptance. The extract can be made into a powder form with some stickiness. The extract activity is relatively stable under 100° C. for at least 30 minutes. The extract is ideal to be incorporated into food products to add on fat-blocking function. Another advantage is that it can be extracted from biomass of sweet potato by-products such as leaves and stems, and hence can have a low production cost.

The present invention also discloses an edible composition, comprising at least one resin glycoside extracted from a plant selected from the Convolvulaceae family as disclosed herein. In some embodiments, the at least one resin glycoside is selected from the group consisting of Batataoside I, Batataoside II, Batatoside III, Pescaprein XXVII, Batatoside F, Batatoside H, a resin glycoside of Formula (I), a resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII). In other embodiments, the edible composition comprises an extract from Ipomoea batatas (sweet potato) and/or Ipomoea aquatica (kangkong). In this regard, the edible composition can comprise an extract from both Ipomoea batatas (sweet potato) and Ipomoea aquatica (kangkong).

In some embodiments, the edible composition further comprises an excipient. The composition may contain any suitable carriers, diluents or excipients. These include all conventional solvents, dispersion media, fillers, solid carriers, coatings, surfactants, isotonic and absorption agents and the like. It will be understood that the compositions of the invention may also include other supplementary physiologically active agents. An excipient is a substance formulated alongside the resin glycoside and can be for the purpose of long-term stabilization, bulking up solid formulations or to confer an enhancement on the active ingredient in the final dosage form, such as facilitating absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. As is known in the art, the selection of appropriate excipients also depends upon the route of administration and the dosage form, as well as the active ingredient and other factors and it is not easily predictable which is advantageous over another.

In some embodiments, the edible composition can comprise a component selected from protein, fiber, starch, cellulose, lipid, dextrin, polyphenolic compound or a combination thereof.

The integrity of the extract can also be protected by means of encapsulation (e.g. spray-drying, coating, extrusion, coacervation and molecular inclusion) at any time during processing, such as before extraction, during extraction, after extraction, during drying, after drying, or after packaging. Some embodiments utilize microencapsulation. With encapsulation, an encasing layer is attained, for example, via molecular, interfacial, colloidal and bulk physicochemical properties of emulsions. The encasement reduces the reactivity of the core with regard to outside environment, for example, oxygen and water. This permits the extension of shelf life of a product in conventional packaging applications. In some embodiments, encapsulation can be used for controlled release of the inner material or core. The encased pulverized product can remain inactive until direct contact with water. Then the water can dissolve the encasement and the active ingredient is able to react with water.

In some embodiments, the encapsulation of the extract can be used to optimize product functionality, particle size and/or create a new product form. Encapsulation can be done with one or more products including, for example, carbohydrates, soy products, dairy products, corn syrup, hydrocolloids, polymers, waxes, fats, vegetable oils, gum arabic, lecithin, sucrose-esters, mono-diglycerides, pectin, K-carbonate, K-bicarbonate, Na-carbonate, Na₃PO₄, K₃PO₄, maltodextrin, glycerine, threitol, erythritol, xylitol, arabitol, ribitol, sorbitol, mannitol, maltitol, maltotriitol, maltotetraitol, lactitol, hydrogenated isomaltulose, hydrogented starch, liposomes, liposomes in sol-gels, shellac, hydrolyzed fats, ethyl cellulose, hydroxy propyl methylcellulose, starches, modified starches, alginate and alginic acid (e.g., sodium alginate), calcium caseinate, calcium polypectate, carboxyl cellulose, carrageenan, cellulose acetate phthalate, cellulose acetate trimellitate, chitosan, corn syrup solids, dextrins, fatty acids, fatty alcohols, gelatin, gellan gums, hydroxy cellulose, hydroxy ethyl cellulose, hydroxy methyl cellulose, hydroxy propyl cellulose, hydroxy propyl ethyl cellulose, hydroxy propyl methyl cellulose, hydroxy propyl methyl cellulose phthalate, lipids, liposomes, low density polyethylene, mono-, di- and tri-glycerides, pectins, phospholipids, polyethylene glycol, polylactic polymers, polylactic co-glycolic polymers, polyvinyl pyrolindone, stearic acid and derivatives, xanthum and proteins, zein, gluten or other agents to protect against environmental elements.

For example, the extract can be encapsulated using sodium alginate. In some embodiments, the extract is encapsulated as spherical particles. The extract can be encapsulated using a microencapsulation method. For example, an electrospray method can be used (FIG. 10). Other methods can include centrifugal extrusion, vibrational nuzzling, spray drying, ionotropic gelation, coacervation-phase separation, interfacial polycondensation, interfacial cross-linking, in situ polymerisation and matrix polymerisation.

Microencapsulation is a process in which tiny particles or droplets are surrounded by a coating to give small capsules. It can be used to incorporate food ingredients, enzymes, cells or other materials on a micro metric scale. Microencapsulation can also be used to enclose solids, liquids, or gases inside a micrometric wall made of hard or soft soluble film, in order to reduce dosing frequency and prevent the degradation of pharmaceuticals. In a relatively simple form, a microcapsule can be a small sphere with a uniform wall around it. The material inside the microcapsule is referred to as the core, internal phase, or fill, whereas the wall is sometimes called a shell, coating, or membrane. Some materials like lipids and polymers, such as alginate, may be used as a mixture to trap the material of interest inside. Most microcapsules have pores with diameters between a few micrometers and a few millimeters.

In another embodiment, the extract is encapsulated using ethyl cellulose, polyvinyl alcohol, gelatin or sodium alginate.

The spherical particles can be of about 100 µm to about 1000 µm in diameter, or about 100 µm to about 800 µm in diameter, or about 100 µm to about 600 µm in diameter, or about 100 µm to about 500 µm in diameter, or about 100 µm to about 300 µm in diameter. In some embodiments, the particles can be about 200 µm in diameter.

In some embodiments, the encapsulation efficiency is of about 60% to about 80%. Encapsulation efficiency is the percentage of an entity that is successfully entrapped into the micelle or nanoparticle. Encapsulation efficiency (EE%) is calculated by (total entity added - free non-entrapped entity) divided by the total entity added. In other embodiments, the EE% is about 65%, about 70%, about 75%, or about 80%.

In some embodiments, the edible composition is provided as a capsule. In other embodiments, the edible composition is provided as a tablet or sachet. Each capsule, tablet or sachet can contain a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

Advantageously, the inventors have found that to maximize the weight loss or weight management function of the edible composition or extract, the resin glycoside should be released only when it is in the intestinal tract as it is at this location that lipid is absorbed into the body. Accordingly, having a ‘barrier’ to prevent the release of resin glycoside and to prevent the degradation of resin glycoside in the presence of stomach acid can allow the edible composition to be more effective.

Some of the advantages of the extract and the edible composition are as follows

| | |
|---|---|
| High lipase inhibition activity | The compound could suppress the digestion and absorption of dietary lipids and restrict energy intake. It has very promising application for developing body weight management products. |
| Extract from edible plants | The compound can be found in edible vegetables makes it readily to be applied into food products, and more acceptable for consumers. |
| Food-grade extraction method | No toxic solvent is needed during extraction process, and solvent can be recycled for further use. The whole method is environmentally friendly and cost efficiency, which also meets the food-grade standards. |
| High yield of the origin plants | Sweet potato and kangkong have strong reproductive capacity and are suitable for mass production for urban farming crop in Singapore. |
| Formulation for controlled-release of lipase inhibitors | Unique formulation that enable the inhibitors to be protected from being degraded in stomach acidic conditions yet rapidly released in small intestine and inhibit lipases. |

The edible composition or extract can be used in weight loss or weight management. In this regard, the subject in need thereof does not have to have a disease or disorder related to excessive body fat. For example, the subject in need thereof does not have to be obese. For example, the subject in need thereof can be in an acceptable body mass index (BMI) range.

The present invention also discloses a pharmaceutical composition comprising resin glycoside from a plant selected from the Convolvulaceae family as disclosed herein, or a pharmaceutically acceptable salt, solvate or isomer thereof, optionally in combination with a pharmaceutically acceptable carrier, excipient or diluent.

The carrier, excipient or diluent must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

It should be understood that in addition to the active ingredients particularly mentioned above, the composition or combination of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The present invention also discloses a method of treating a disease or disorder associated with excessive body fat in a subject in need thereof, comprising administering at least one resin glycoside from a plant selected from the Convolvulaceae family as disclosed herein.

In some embodiments, the method of treating a disease or disorder associated with excessive body fat in a subject in need thereof, comprising administering resin glycoside of Formula (I).

In some embodiments, the method of treating a disease or disorder associated with excessive body fat in a subject in need thereof, comprising administering resin glycoside of Formula (II).

In some embodiments, the method of treating a disease or disorder associated with excessive body fat in a subject in need thereof, comprising administering resin glycoside of Formula (III).

In some embodiments, the method of treating a disease or disorder associated with excessive body fat in a subject in need thereof, comprising administering resin glycoside of Formula (IV).

In some embodiments, the method of treating a disease or disorder associated with excessive body fat in a subject in need thereof, comprising administering resin glycoside of Formula (V).

In some embodiments, the method of treating a disease or disorder associated with excessive body fat in a subject in need thereof, comprising administering resin glycoside of Formula (VI).

In some embodiments, the method of treating a disease or disorder associated with excessive body fat in a subject in need thereof, comprising administering resin glycoside of Formula (VII).

As used herein, the method of treating a disease or disorder associated with excessive body fat also includes a method of preventing excessive body fat accumulation.

In some embodiments, the disease or disorder is obesity. Obesity is a complex disease involving an excessive amount of body fat. It is a medical problem that increases your risk of other diseases and health problems, such as heart disease, diabetes, high blood pressure and certain cancers. For example, obesity can be diagnosed when a subject’s body mass index (BMI) is 30 or higher. To determine body mass index, divide weight in kilograms by height in meters squared. In this regard, in some embodiments, the method of treating obesity comprises treating a subject with a BMI value of more than 30.

The disease or disorder can be overweight. Overweight is generally due to extra body fat. A person with a BMI of 25-29.9 is considered overweight.

In some embodiments, the disease or disorder is a BMI of more than 25.

In some embodiments, the disease or disorder is a metabolic syndrome. In other embodiments, the metabolic syndrome is associated with obesity, and includes, but is not limited to, insulin resistance, dyslipidemia, and elevated human C-reactive protein (CRP) levels. Other metabolic syndromes can be hypertension, stroke, heart disease, diabetes, peripheral vascular disease, cardiac hypertrophy and congestive heart failure.

In other embodiments, the method of treating a disease or disorder associated with excessive body fat is a method for weight loss or a method for weight management.

The present invention also discloses a use of an extract for treating a disease or disorder associated with excessive body fat in a subject in need thereof, the extract comprising resin glycoside from a plant selected from the Convolvulaceae family as disclosed herein.

In some embodiments, the use is a use of a resin glycoside of Formula (I) for treating a disease or disorder associated with excessive body fat in a subject in need thereof.

In some embodiments, the use is a use of a resin glycoside of Formula (II) for treating a disease or disorder associated with excessive body fat in a subject in need thereof.

In some embodiments, the use is a use of a resin glycoside of Formula (III) for treating a disease or disorder associated with excessive body fat in a subject in need thereof.

In some embodiments, the use is a use of a resin glycoside of Formula (IV) for treating a disease or disorder associated with excessive body fat in a subject in need thereof.

In some embodiments, the use is a use of a resin glycoside of Formula (V) for treating a disease or disorder associated with excessive body fat in a subject in need thereof.

In some embodiments, the use is a use of a resin glycoside of Formula (VI) for treating a disease or disorder associated with excessive body fat in a subject in need thereof.

In some embodiments, the use is a use of a resin glycoside of Formula (VII) for treating a disease or disorder associated with excessive body fat in a subject in need thereof.

The present invention also discloses a use of an extract in a manufacture of a medicament for treating a disease or disorder associated with excessive body fat, the extract comprising resin glycoside from a plant selected from the Convolvulaceae family as disclosed herein.

In some embodiments, the use is a use of a resin glycoside of Formula (I) in a manufacture of a medicament for treating a disease or disorder associated with excessive body fat.

In some embodiments, the use is a use of a resin glycoside of Formula (II) in a manufacture of a medicament for treating a disease or disorder associated with excessive body fat.

In some embodiments, the use is a use of a resin glycoside of Formula (III) in a manufacture of a medicament for treating a disease or disorder associated with excessive body fat.

In some embodiments, the use is a use of a resin glycoside of Formula (IV) in a manufacture of a medicament for treating a disease or disorder associated with excessive body fat.

In some embodiments, the use is a use of a resin glycoside of Formula (V) in a manufacture of a medicament for treating a disease or disorder associated with excessive body fat.

In some embodiments, the use is a use of a resin glycoside of Formula (VI) in a manufacture of a medicament for treating a disease or disorder associated with excessive body fat.

In some embodiments, the use is a use of a resin glycoside of Formula (VII) in a manufacture of a medicament for treating a disease or disorder associated with excessive body fat.

### EXAMPLES

General Experimental Procedures. Light absorbance data was measured on 96-well microplates (Corning, clear polystyrene) in a microplate reader (Synergy HT, Biotek Instruments Inc., Winooski, VT, USA) at 410 nm. Column chromatography (CC) was carried out using silica gel (40-63 µm, Merck, Germany). The instrumentation used for HPLC analysis consisted of a Waters (Millipore Corp., Waters Chromatography Division, Milford, MA) Alliance 2695 separations module equipped with a Waters 2996 PDA (UV/Vis) detector and a reversed-phase C₁₈ column (Phenomenex, 5 µm, 4.6×250 mm). MS spectra were acquired using a Finnigan/MAT LCQ ion trap mass spectrometer (San Jose, CA, USA) equipped with an electrospray ionization (ESI) source. LC-ESI/MS² were acquired using a Bruker Amazon ion trap mass spectrometer (Billerica, MA, USA) equipped with a Dionex ultimate 3000RS LC system (Bannockburn, IL, USA). ¹H NMR spectra was recorded with a Bruker AV500 spectrometer (Karlsruhe, Germany) at 500 MHz. Removal of the solvents were conducted by a rotatory evaporator equipped with a vacuum pump (Rotavapor® R-100, V-100, I-100, BUCHI, Switzerland). All solvents were of analytical grade, except those used in HPLC analysis were of spectrum grade.

Plant Materials. Tubers of I. batatas (400 g) were purchased in a local market while leaves of I.batatas (2.1 kg) was purchased from a local farm in Singapore. Leaves of I. aquatica (2.092 kg) (Pasar, Malaysia) were purchased from a grocery (Fairprice). The tubers and leaves were washed with de-ionized water and stored in freezer (-18° C.) for further tests.

Extraction and Fractionation (Step a and b). The tubers (400 g) were homogenized by a mixer with addition of ethanol (1.5 L), then extracted by maceration at room temperature (23° C.) for 24 hours. The extract was filtered through a Büchner funnel (150 mL, Synthware, Beijing, China) and concentrated to 30 mL with rotatory evaporator. The concentrated extract was extracted by 25 mL ethyl acetate for 3 times, upper-layer was collected, combined, and dried by rotatory evaporator to give a dark-brown syrup (1.7 g).

The syrup was further purified by silica gel column chromatography. On a column with 20 g silica gel, 1.0 g dried sample was loaded, and eluted using a gradient from hexane-ethyl acetate (1:1) to pure ethyl acetate. Pure methanol was then eluted for 100 mL to wash out the residue compounds on column. Monitored by thin-layer chromatography, totally 5 fractions (F1-F5) were collected, and PL inhibition activity were tested for each fraction. The fraction F5 eluted with methanol was further purified on a silica gel column with a gradient from DCM-MeOH (7:1) to pure MeOH. Totally 5 fractions (F5.1-F5.5) were collected and tested activity. Fraction F5.1 was proceed for further purification by preparative thin layer chromatography (PTLC), with developing reagent of DCM-MeOH (7:1). Totally 5 fractions (F5.1.1-F5.1.5) were collected and tested activity. Fraction F5.1.2 and F5.1.3 were combined (F5.1.23, 216 mg) and stored in freezer (-18° C.) until its PL inhibition activity was tested.

The leaves of I. batatas (9.2 kg) were homogenized by a blender before being percolated with dichloromethane (20 L) at room temperature (23° C.) for 24 hours. The extract was then filtered through a Büchner funnel (150 mL, Synthware, Beijing, China) and concentrated using a rotary evaporator to obtain a dark-green syrup (25.5 g). The dark green-syrup was than extracted by methanol (1.275 L) and the supernatant was collected and dried using the rotary evaporator to give a dry extract (20.6 g). This dry extract was subsequently dissolved in methanol and partitioned between hexane: methanol: water in the ratio of 1:1:0.05 (v/v/v). The lower layer was then concentrated to give a brown residue (12.7 g).

The brown residue (9 g) was further purified using Silica gel column chromatography. On a column with 60 g of silica gel, a total of 3 g of sample was loaded and eluted using a gradient of hexane and ethyl acetate (5:1), (1:1), (0:1). Pure methanol was then eluted for 300 mL to wash out the residual compounds on column. In total 4 different fractions were obtained (CC1-CC4), and PL inhibition activity were tested for each fraction.

The leaves of I. aquatica (2.092 kg) were homogenized by a blender with the addition of dichloromethane (4 L) at room temperature (23° C.) for 24 hours. The extract was then filtered through a Büchner funnel (150 mL, Synthware, Beijing, China) and concentrated using a rotary evaporator to obtain a dark-green syrup (2.95 g). This extract (2.95 g, 200 mg/mL) was dissolved in methanol and purified using Silica gel column chromatography with a gradient of hexane and ethyl aetate (5:1, 1:1, 0:1). Pure methanol was then utilised to eluted out all residual compounds.

Bioassay-based Isolation of Pancreatic Lipase (PL) Inhibitors (Step c). The HPLC analysis were done on an analytical reversed-phase C₁₈ column (Phenomenex, 5 µm, 4.6×250 mm), flow rate of 1.0 mL/min, and sample injection of 10 µL (50 mg/mL) at 30° C. column temperature. The PDA detector was set to scan from 190 to 400 nm, resolution at 1.2 nm, and peaks were monitored at 210 and 280 nm. F5.1.23 and CC1-CC4 were eluted using this protocol. The gradient elution was from MeOH—H₂O (90:10) to MeOH-H₂O (100:0), and fractions was collected every minute. Each fraction was tested PL inhibition activity. LC-ESI/MS was conducted with same elution, and each peak in UV spectra was identified. Then semi-preparative HPLC separation was done with same elution pattern, with a flow rate of 4.71 mL/min. Fraction 3 (F5.1.23.3) and 4 (F5.1.23.4) of sweet potato tuber were further purified with isocratic elution of pure CH₃CN to afford Batataoside I (1.1 mg), Batataoside II (0.9 mg), Batatoside III (0.8 mg) and Pescaprein XXVII (0.7 mg). Similarly, Fraction 8 (F5.1.23.8) and 10 (F5.1.23.10) were further purified with an isocratic elution of ACN:MeOH (80:20) to obtain Batatoside F (5.15 mg) and Batatoside H (1.62 mg) respectively.

This process can also be similarly applied to Kangkong whereby a series of resin glycosides in the methanolic fraction from silica column chromatography were characterized.

Pancreatic Lipase Activity Assay Preparation The pancreatic lipase activity assay uses 4-nitrophenyl palmitate (pNPP) as colorimetric lipase substrate. Pancreatic lipase (from porcine pancreas, type II, 100-500 units/mg, L3126), sodium deoxycholate (D6750), 4-nitrophenyl palmitate (N2752), and Orlistat (PHR1445) were obtained from Sigma-Aldrich (St Louis, MO, USA). Tris-Borate-EDTA buffer (10X, pH 8.3, PB 1040) was purchased from Vivantis Technologies (Selangor, Malaysia).

Buffer solution was prepared by diluting the 10X Tris-Borate-EDTA buffer (890 mM) into 50 mM solution, followed by addition of 0.35% (m/v) sodium deoxycholate, and adjust pH to 8.3. pNPP solution was prepared by adding pNPP into isopropanol to make a 1.5 mg/mL solution. Both solutions were stored in fridge (4° C.) for further tests. PL solution was prepared by suspending PL in 4° C. buffer solution (10 mg/mL), shaking by vortex mixer for 2 min, and centrifuging at 4° C., 6000 rpm for 5 min. The supernatant was divided into several 0.5 mL aliquots in 0.6 mL PP centrifuge tubes and stored in freezer (-18° C.) for further tests.

High-throughput Pancreatic Lipase Activity Assay. For each sample, 20 µL of extract was transferred into a 0.6 mL PP centrifuge tube, dry up at room temperature for 10 min, followed by adding 300 µL buffer solution. The mixture was sonicated thoroughly in water bath sonicator, 150 µL of the sample was transferred onto a 96-well microplate (Corning, clear polystyrene, USA), and incubated in microplate reader (Synergy HT, Biotek Instruments Inc., Winooski, VT, USA) at 37° C. for 15 min. Meanwhile, frozen lipase solution was thawed in room temperature, 10 µL of which was added into the well, and incubate at 37° C. for another 10 min. Finally, the reaction starts by adding 10 µL pNPP solution into each well. The reading wavelength was fixed at 410 nm, 3 s shaking before each reading, and a 1-hour kinetic data was obtained by a reading interval of 20 or 60 seconds. Negative control group (blank) was prepared by adding 20 µL pure solvent instead of extract in the first step.

The tendency of readings in the first 3 minutes were regarded as linear growth, and the slope was calculated by linear regression in Excel (Office 2016, Microsoft, USA). The inhibition activity of each sample is calculated in percentage inhibition by Eq. 1.

$$\text{Inhibition} \left(\%\right) = 1 - \left({\text{slope}}_{\text{sample}} / {\text{slope}}_{\text{control}}\right) \times 100 \% \qquad \text{(1)}$$

Twelve sweet potato tuber cultivars on the market were screened with the method described above. The inhibitory activity towards PL were tested with crude extract solution which was diluted 5, 10 and 20 times (Table 0).

TABLE 0

Screening PL inhibition of twelve sweet potato tuber cultivars with 5x, 10x and 20x diluted extract

| Dil. | Tubers (% Inhibition) | | | | | | | | | | | |
|---|---|---|---|---|---|---|---|---|---|---|---|---|
| | A | B | C | D | E | F | G | H | I | J | K | L* |
| 5x | 48.95 | 54.00 | 50.18 | 57.49 | 40.14 | 57.49 | 49.60 | 47.56 | 50.66 | 48.62 | 51.99 | 54.26 |
| 10x | 38.31 | 43.92 | 39.36 | 41.50 | 35.83 | 51.51 | 39.36 | 41.81 | 34.92 | 36.60 | 38.38 | 42.04 |
| 20x | 22.90 | 29.96 | 28.67 | 40.78 | 32.13 | 51.12 | 28.90 | 43.12 | 25.17 | 25.27 | 36.44 | 39.71 |

* A) Vietnam Purple; B) Indonesia Honey; C) Japan Kanta; D) Indonesia Orange; E) Korea Pumpkin; F) Vietnam Japanese; G) Japan Silk/Hakura; H) Indonesia Honey; I) America Covington; J) Australia Gold; K) Indonesia Purple; L) Indonesia Red;

### Encapsulation of Resin Glycosides Extracted From Vegetable

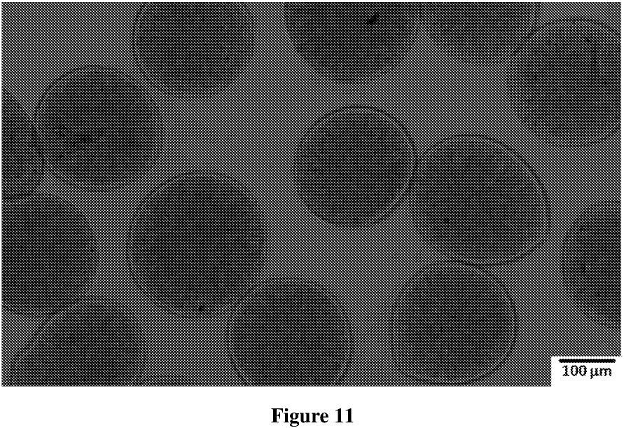

Generally, the encapsulation is carried out by electrospray method according to FIG. 10. The crude extract is dispersed into a 0.5% (w/v) aqueous solution of sodium alginate, making a final concentration at 1 mg/mL. The extract solution is loaded into a 50 mL syringe connected with a needle. The syringe is placed onto a syringe pump to provide a steady flow at 2.0 mL/min. A high voltage supply provides 6.0 kV voltage, with the anode connected to the syringe needle, and cathode connected to and ring-shape electrode. The sprayed droplets are collected by 0.1 mol/L calcium chloride solution in a beaker and kept in the solution for 30 minutes with constant stirring. The encapsulated particles have an average diameter at around 200 µm, as seen in FIG. 11. The encapsulation efficiency (amount of extract in the initial solution/amount of extract in the capsules) is 74%.

The pH responsive release of lipase inhibitors extracted from vegetables and from its encapsulated form was studied. Both solution form and encapsulated form of the extract is incubated at 37° C. in a simulated gastric fluid at pH 3 for 4 hours, followed by a simulated intestinal fluid at pH 7 for 2 hours. Of 15 µg/mL final concentration of the extract, inhibition of solution form dropped from 34% to 14%, while inhibition of encapsulated form dropped from 37% to 34%. The encapsulation process provided protection for the extract against acidic condition in gastric phase.

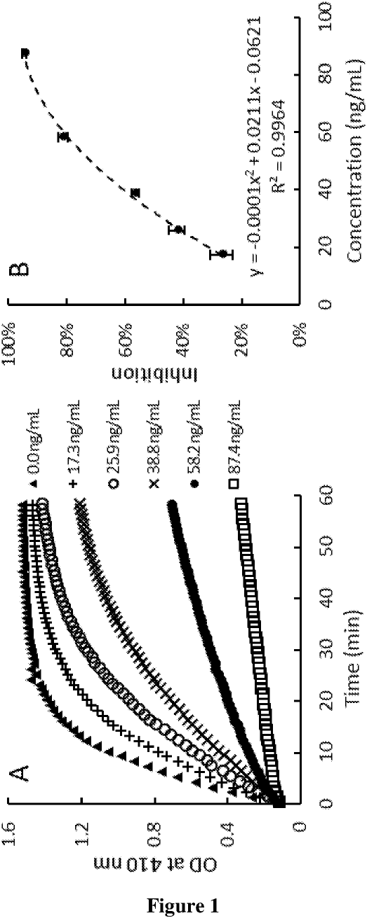

Comparator PL Inhibitory Assay (pNPP as substrate). p-Nitrophenyl palmitate (pNPP) is a synthetic chemical probe widely used in PL inhibitory assay. Long chain fatty acid (palmitic acid) and chromatic indicator (p-nitrophenol) are linked by an ester bond, and the molecule works as a substrate of PL. Hydrolyzed by PL in buffer solution, pNPP molecules release the indicators, and a yellowish color is presented. PL activity is indicated by the speed of color gain by time, and the color could be measured by optical density at 410 nm by a spectrometer. FIG. 1A showed a representative graph of PL inhibitory assay by pNPP using Orlistat as inhibitor, which is a commercially available lipase inhibitor drug. According to Michaelis-Menten equation, PL activity could be presented by the initial rate of the reaction. In the present study, initial slope of the curve could be considered as initial reaction rate, and the data in the first 5 minutes could be considered as linear (R²>0.999) in most cases. The inhibition is calculated percentage by Eq.1, and a dose-response curve could be plotted according to PL inhibition under different inhibitor concentrations (FIG. 1B). Fitting with proper model, IC₅₀ could be calculated by plugging 50% into the fitted formula.

Bioassay-based Fractionation of PL Inhibitors in sweet potato tuber. The ethanol extract of sweet potato showed lipase inhibition with IC₅₀ of 33 µg/mL. Further two rounds of fractionation (purification) with silica gel column chromatography and PTLC followed by inhibition activity test showed that F5, F5.1, and F5.1.23 had significant PL inhibition.

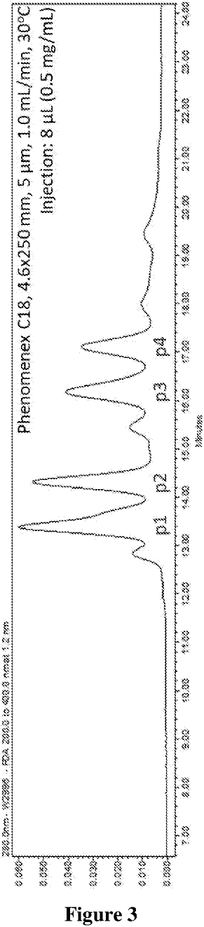

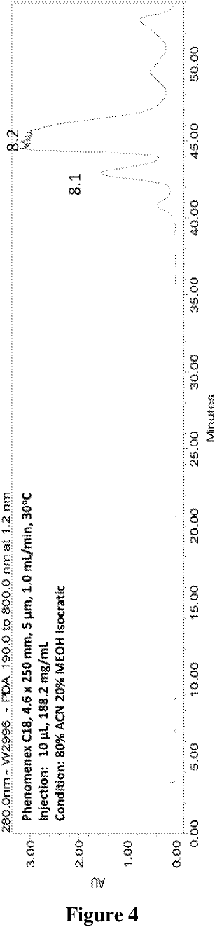

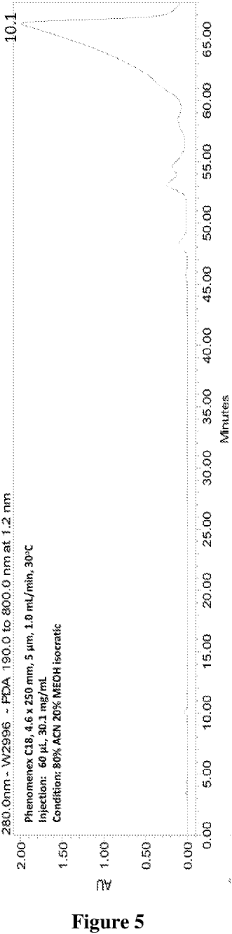

HPLC Isolation and Identification of PL Inhibitors in sweet potato tuber. Further isolation of the active fraction F5.1.23 was done by two rounds of HPLC separation, first round using MeOH—H₂O elution to fractionate each peak roughly, and second round using CH₃CN elution peak by peak, to isolate pure compounds (Step c). The chromatogram of first round HPLC analysis is shown on FIG. 2 overlapping with relative lipase activity in orange line. The active compounds were mainly concentrated at retention time 10-15 and 20-24 minutes. Fraction 3 and 4 (retention time 13.5-15.0 min, combined as F5.1.23.34) of first round HPLC separation were further purified by HPLC using pure CH₃CN elution, and 4 peaks (p1-p4) were collected, characterized, and tested for lipase inhibition activity (FIG. 3, Table 1). Additionally, both Fractions 8 (Retention time 21.0-21.5 min) and 10 (Retention time 23.0-24.0) were further purified by HPLC using an isocratic elution of ACN:MeOH (80:20). During the HPLC separation, 2 resin glycosides from Fraction 8 (FIG. 4, Table 2) and 1 resin glycoside from Fraction 10 (FIG. 5, Table 2) were isolated and tested for lipase inhibition activity respectively.

Based on 1.1016 g of crude extract, 188.2 mg of fraction 8 can be obtained followed by 45.5 mg of fraction 5 and 30.1 mg of fraction 10. The yield for Batatoside I, Batatoside II, Batatoside III and Prescaprein XXVII is about 16.5, 7.87, 11.9, 7.11% (w/w) when extracted from Fraction 5 respectively. On the other hand, 16.1% (w/w) of Batatoside F can be obtained from Fraction 8. 22.4% (w/w) of Batatoside H can be obtained from Fraction 10. Therefore, the highest concentration of resin glycoside, Batatoside F (30 mg) can be obtained from the same amount of crude extract, followed by Batatoside H (6.7 mg), Batatoside I (7.5 mg), Batatoside III (5.4 mg), Batatoside II (3.6 mg) and Prescaprein XXVII (3.2 mg) respectively.

TABLE 1

Observed masses, possible structures and lipase inhibition activity of resin glycosides in fraction F5.1.23.34.

| | |
|---|---|
| 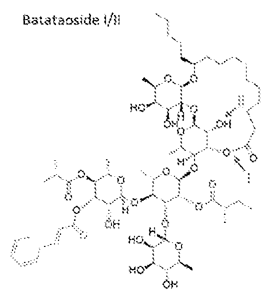 | 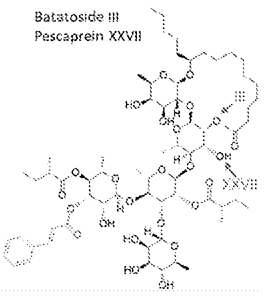 |

| | | | | | |
|---|---|---|---|---|---|
| Peak | Structural assignment | m/z (-) | Weight (mg) | IC₅₀ (µg/mL) | IC₅₀ (µM) |
| p1 | Batataoside I | 1267.6 | 1.1 | 8.5 | 6.7 |
| p2 | Batataoside II | 1267.6 | 0.9 | 7.6 | 6.0 |
| p3 | Batatoside III | 1281.5 | 0.8 | 6.3 | 4.9 |
| p4 | Pescaprein XXVII | 1281.5 | 0.7 | 7.2 | 5.6 |

TABLE 2

Observed masses, possible structures and lipase inhibition activity of resin glycosides in Fraction 8 and 10

| Fraction 8.1 | | Fraction 8.2 | | Fraction 10.1 | | |
|---|---|---|---|---|---|---|
| 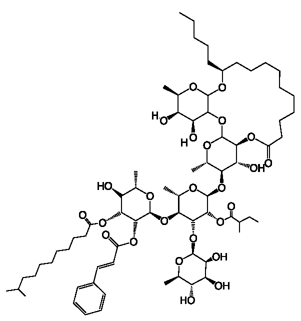 | | 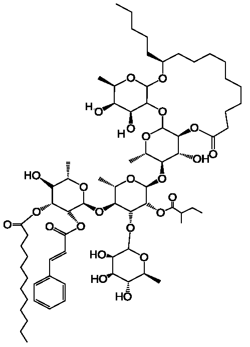 | | 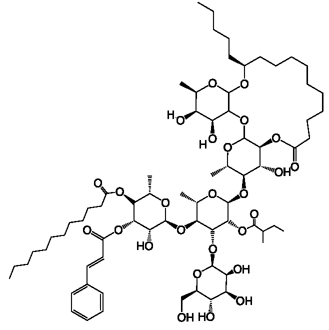 | | |
| 8.1 | Formula (I) | 1379 | 0.94 | | 8.97 ± 0.74 | 6.50 ± 0.54 |
| 8.2 | Batatoside F | 1379 | 5.15 | | 10.87 ± 1.71 | 7.88 ± 1.24 |
| 10.1 | Batatoside H | 1395 | 1.62 | | 14.80 ± 0.79 | 10.23 ± 0.34 |

PL Inhibitory Activity of Resin Glycosides in Sweet potato tuber. After two rounds of HPLC isolation and purification, totally Batataoside I (1.1 mg), Batataoside II (0.9 mg), Batatoside III (0.8 mg), Pescaprein XXVII (0.7 mg), Batatoside F (5.15 mg) and Batatoside H (1.62 mg) were collected. PL inhibition potency of these resin glycosides was tested along with Orlistat as positive control (Table 1), and IC₅₀ were among 4.9 to 10.6 µM for resin glycosides, and 64.4 nM for Orlistat respectively.

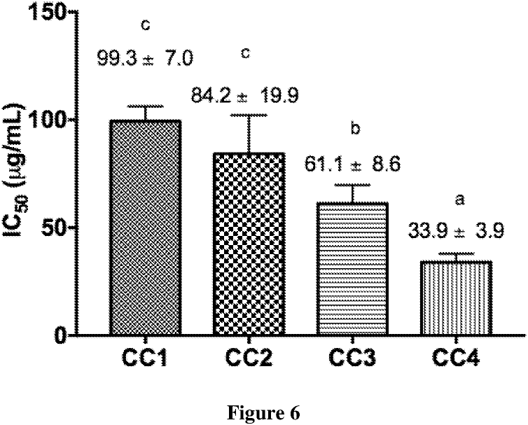

PL inhibitory assay for Sweet potato leaves. The DCM extract of the sweet potato leaves showed a lipase inhibition with IC₅₀ of 90.9 ± 20.2 µg/mL. Further fractionation with methanol achieved a lower IC₅₀ of 59.5 ± 27.5 µg/mL. Liquid-liquid fractionation was carried out with 1:1:0.05 (v/v/v) Hexane: methanol: deionized water which yield an IC₅₀ of 47.4 ± 2.0 µg/mL. Further fractionation with silica gel chromatography yield four fractions, CC1-CC4 and CC4 showed the highest inhibition activity, IC₅₀= 33.9 ± 3.9 µg/mL (FIG. 6).

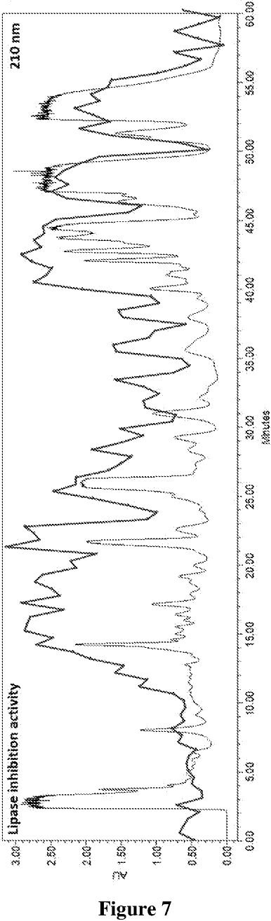

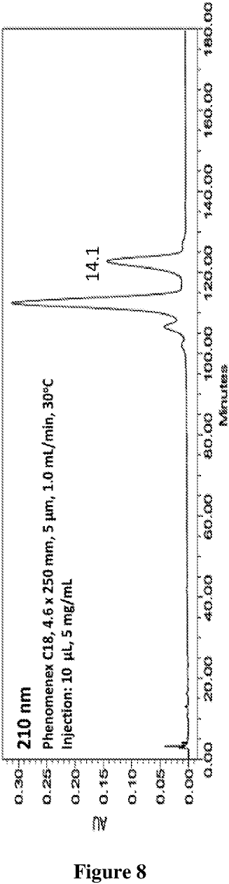

HPLC Isolation and Identification of PL Inhibitors in Sweet potato leaves. Further isolation of the active fraction CC4 was done by HPLC separation using MeOH—H₂O elution to fractionate each peaks. The chromatogram of HPLC analysis is shown on FIG. 7 overlapping with relative lipase activity in red line. The active compounds were mainly concentrated at retention time 11-30 and 40-55 minutes. For the identification of compounds in CC4, LC-ESI/MS² analysis was applied using the same elution protocol and a series of resin glycosides were proposed (Table 3). Fraction 14 (retention time 55.0 to 60.0 min) from first round of separation was further purified and fraction 14.1 was collected and characterised (FIG. 8, Table 4).

TABLE 3

Observed masses and proposed structures of resin glycosides in CC4 of sweet potato leaves

| 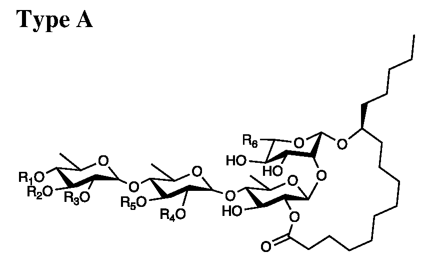 | | | | | | 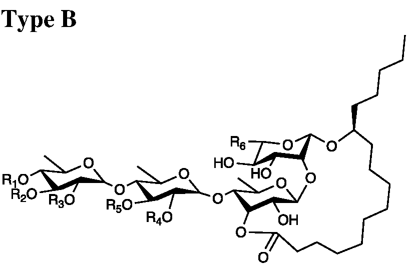 | | | | | |
|---|---|---|---|---|---|---|---|---|---|---|---|
| Retention time | (-) MS (m/z) | (-) MS² (m/z) | Proposed compound | Molecular formula | R₁ | R₂ | R₃ | R₄ | R₅ | R₆ | Type |
| 16.5 | 1297 | 433, 563, 836, 937, 1149 | Cairicoside I | C₆₅H₁₀₂O₂₆ | Mba | Cna | H | Mba | Rha | CH₂OH | A |
| | 1297 | 433, 563, 836, 937, 1149 | Cairicoside A | C₆₅H₁₀₂O₂₆ | Mba | Cna | H | Mba | Rha | CH₂OH | B |
| 18.4 | 1267 | 277, 417, 547, 921, 1167, 1139, 1119 | Batataoside II | C₆₄H₁₀₀O₂₅ | Iba | Cna | H | Mba | Rha | CH₃ | A |
| | 1267 | 277, 417, 547, 921, 1167, 1139, 1119 | Batataoside I | C₆₄H₁₀₀O₂₅ | Iba | Cna | H | Mba | Rha | CH₃ | B |
| | 1267 | 277, 417, 547, 921, 1167, 1139, 1119 | Batatoside A | C₆₄H₁₀₀O₂₅ | H | Iba | Cna | Mba | Rha | CH₃ | B |
| 18.7 | 1437 | 417, 547, 734, 837, 1255, 1290, 1293, 1311 | Intrapilosin VII | C₇₅H₁₂₂O₂₆ | Octa | Cna | H | Dodeca | Glu | CH₃ | A |
| 24.4 | 1223 | 417, 545, 1051, 1125 | Murucoidin XIX | C₆₀H₁₀₄O₂₅ | Ace | H | H | Dodeca | Rha | CH₃ | A |
| 28.4 | 1339 | 417, 545, 1093, 1191, 1213 | Intrapilosin II | C₆₈H₁₀₈O₂₆ | Octa | Cna | H | Mba | Glu | CH₃ | A |
| 29.4 | 1383 | 433, 563, 1082, 1165, 1229,1300 | Acutacoside C | C₇₀H₁₁₂O₂₇ | Deca | Cna | H | Mba | Glu | CH₂OH | A |
| 36.6 | 1249 | 417, 545, 1019, 1067, 1049 | Pescaprein III | C₆₃H₁₁₀O₂₄ | Mba | H | H | Dodeca | Rha | CH₃ | B |
| | 1249 | 417, 545, 1019, 1067, 1049 | Batatinoside VIII | C₆₃H₁₁₀O₂₄ | H | Mba | H | Dodeca | Rha | CH₃ | B |
| | 1249 | 417, 545, 1019, 1067, 1049 | Stoloniferin X | C₆₃H₁₁₀O₂₄ | Mba | H | H | Dodeca | Rha | CH₃ | A |
| 37.0 | 1367 | 417, 545, 1213, 1066 | Acutacoside A | C₇₀H₁₁₂O₂₆ | Mba | Cna | H | Deca | Glu | CH₃ | A |
| | 1367 | 417, 545, 1213, 1066 | Purginoside I | C₇₀H₁₁₂O₂₆ | Mba | H | Cna | Deca | Glu | CH₃ | A |
| 39.2 | 1395 | 433, 536, 1213, 1247,1293 | Batatoside M | C₇₂H₁₁₆O₂₆ | Mba | H | Cna | Dodeca | Rha | CH₂OH | A |
| 40.5 | 1411 | 433, 563, 1229, 1180 | Acutatoside E | C₇₂H₁₁₆O₂₇ | Dodeca | Cna | H | Mba | Glu | CH₂OH | A |
| | 1411 | 433, 563, 1229, 1180 | Acutatoside D | C₇₂H₁₁₆O₂₇ | Dodeca | H | Cna | Mba | Glu | CH₂OH | A |
| 41.1 | 1381 | 417, 545, 1255, 1135,1108 | Intrapilosin III | C₇₁H₁₁₄O₂₆ | Octa | Cna | H | Octa | Glu | CH₃ | A |
| 42.5 | 1379 | 417, 545, 921, 1278, 1197, 1121 | Batatoside F | C₇₂H₁₁₆O₂₅ | H | Dodeca | Cna | Mba | Rha | CH₃ | A |
| | 1379 | 417, 545, 921, 1278, 1197, 1121 | Batatoside E | C₇₂H₁₁₆O₂₅ | Dodeca | H | Cna | Mba | Rha | CH₃ | A |
| | 1379 | 417, 545, 921, 1278, 1197, 1121 | Batatoside D | C₇₂H₁₁₆O₂₅ | Dodeca | Cna | H | Mba | Rha | CH₃ | A |
| | 1379 | 417, 545, 921, 1278, 1197, 1121 | Batatoside C | C₇₂H₁₁₆O₂₅ | H | Dodeca | Cna | Mba | Rha | CH₃ | B |
| | 1379 | 417, 545, 921, 1278, 1197, 1121 | Batatinoside I | C₇₂H₁₁₆O₂₅ | Dodeca | Cna | H | Mba | Rha | CH₃ | B |
| 43.5 | 1395 | 417, 547, 982, 1066, 1294, 1311 | Batatoside I | C₇₂H₁₁₆O₂₆ | Dodeca | H | Cna | Mba | Glu | CH₃ | B |
| 44.6 | 1409 | 417, 547, 963, 1213, 1096, 1121 | Wolcottinoside IV | C₇₃H₁₁₈O₂₆ | Octa | Cna | H | Deca | Glu | CH₃ | A |
| 53.8 | 1303 | 417, 547, 845, 1121, 1149 | Ipomotaoside D | C₇₁H₁₁₆O₂₁ | Dodeca | H | Cna | H | Deca | CH₃ | B |

*Mba: 2-methylbutanoyl, Dodeca: n-dodecanoyl, Octa: n-octanoyl, Deca: n-decanoyl, Cna: cinnamoyl, Rha: Rhamnopyranosyl, Glu: Glucopyranosyl; Iba: Isobutanoyl, Ace: Acetyl

TABLE 4

Observed mass and proposed structure of resin glycoside in Fraction 14 of sweet potato leaves

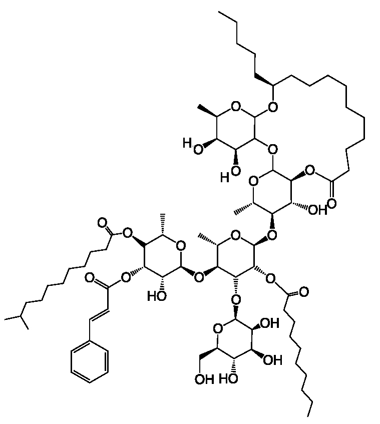

| Peak | Structural assignment | m/z (-) | IC₅₀ (µg/mL) | IC₅₀ (µM) |
|---|---|---|---|---|
| 14.1 | Formula (II) | 1465 | 202.5 ± 52.3 | 138.1 ± 35.7 |

Characterisation of compounds in Sweet potato leaves. LC-ESI/MS² analysis of CC4 with high lipase inhibition activity revealed a series of resin glycoside compounds in the Sweet potato leaves. Overall, the main structure of the resin glycoside in the sweet potato leaves is a pentasaccharide, containing one glucose, fucose and three rhamnose linked to different types of fatty acids (octanoic (caprylic), decanoic (capric), dodecanoic (lauric) and trans-cinnamic acid). After two rounds of HPLC isolation and characterisation process, a new resin glycoside was obtained from Fraction 14 of sweet potato leaves. The PL inhibition activity of this new compound is 138.1 ± 35.7 µM.

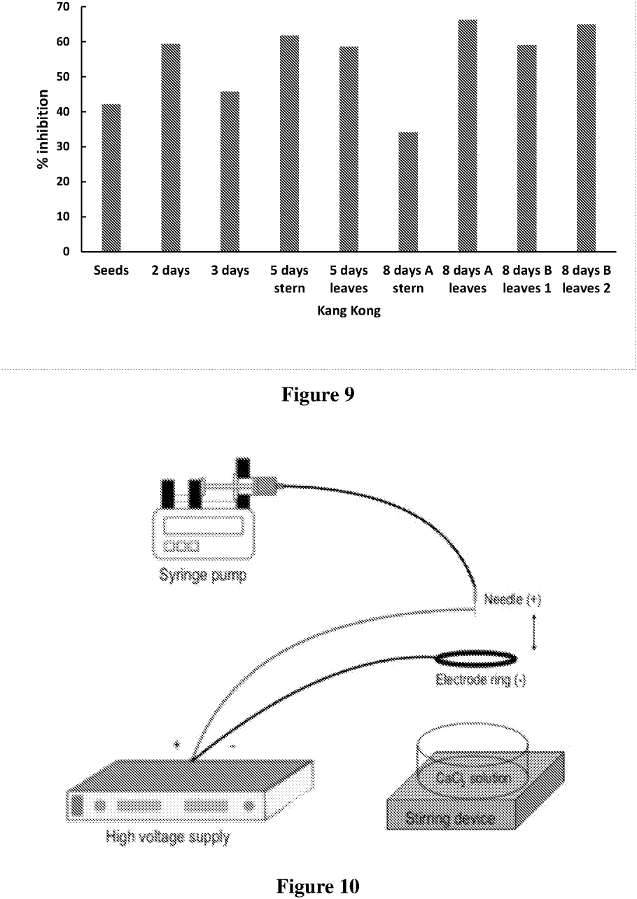

Screening of lipase inhibition activity in kangkong. The plant materials include seed of Kang Kong, whole KangKong with different cultivation time (2 days, 3 days), the leaves and stems of KangKong with different cultivation time (5 days, 8 days). The samples were lyophilized and stored in freezer for further use. The sample was homogenized by a blender before being percolated with dichloromethane at room temperature (23° C.) for 24 hours. The extract was then filtered through a Büchner funnel. The solvent was removed by rotary evaporator and crude extracts were obtained. The lipase inhibitory activity of samples were evaluated at concentration of 0.1 mg/mL. As shown in FIG. 9, all extracts have lipase inhibitory effects. Extracts from germinated KangKong are more effective than that of seed. Besides, no relationship was found between cultivation time and lipase inhibitory effects.

PL inhibitory assay for Kang Kong from Pasar. The DCM extract of the Kang Kong from Pasar showed a lipase inhibition with IC₅₀ of 652.80 µg/mL. Further fractionation with methanol achieved a lower IC₅₀ of 113.81 µg/mL. After methanol extraction, silica gel chromatography was carried out and the methanolic fraction yield an increased pancreatic lipase inhibition with an IC₅₀ value of 59.38 µg/mL.

HPLC-ESI-MS/MS Identification of PL Inhibitors in Kang Kong. Further isolation of the active methanolic fraction was done by HPLC separation using MeOH—H₂O elution to fractionate each peaks. The peaks were eluted between 18 min to 30 min. LC-ESI/MS² analysis was applied using the same elution protocol and a series of resin glycosides were proposed (Table 5).

TABLE 5

Observed masses and proposed structures of resin glycosides in Kang Kong

| Type A | | | | | | Type B | | | | | |
|---|---|---|---|---|---|---|---|---|---|---|---|
| 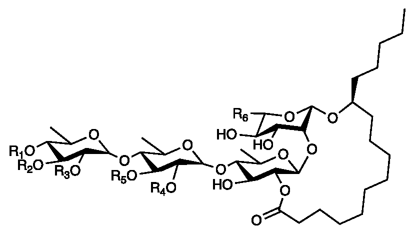 | | | | | | 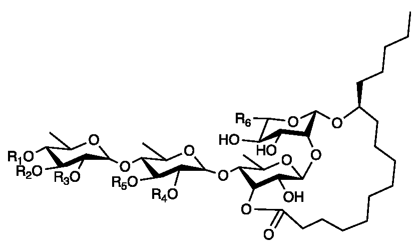 | | | | | |
| Retention time/min | (-) MS (m/z) | (-) MS² (m/z) | Proposed compound | Molecular formula | R₁ | R₂ | R₃ | R₄ | R₅ | R₆ | Type |
| 13.6 | 1335 | 417, 543, 963, 1065, 1191, 1336 | Formula (III) | C₆₇H₁₁₆O₂₆ | Dodeca | Cna | Mba | H | Rha | CH₃ | A |
| 16.3 | 1379 | 417, 543, 946, 1089, 1251, 1379 | Batatoside F | C₇₂H₁₁₆O₂₅ | H | Dodeca | Cna | Mba | Rha | CH₃ | A |
| | 1379 | 417, 543, 946, 1089, 1251, 1379 | Batatoside E | C₇₂H₁₁₆O₂₅ | Dodeca | H | Cna | Mba | Rha | CH₃ | A |
| | 1379 | 417, 543, 946, 1089, 1251, 1379 | Batatoside D | C₇₂H₁₁₆O₂₅ | Dodeca | Cna | H | Mba | Rha | CH₃ | A |
| | 1379 | 417, 543, 946, 1089, 1251, 1379 | Batatoside C | C₇₂H₁₁₆O₂₅ | H | Dodeca | Cna | Mba | Rha | CH₃ | B |
| | 1379 | 417, 543, 946, 1089, 1251, 1379 | Batatinoside I | C₇₂H₁₁₆O₂₅ | Dodeca | Cna | H | Mba | Rha | CH₃ | B |
| 17.7 | 1235 | 417, 981, 1091, 1235 | Aquaterin VII | C₆₂H₁₀₈O₂₄ | Octa | H | H | Octa | Rha | CH₃ | B |
| | 1235 | 417, 981, 1091, 1235 | Aquaterin VI | C₆₂H₁₀₈O₂₄ | H | Octa | H | Octa | Rha | CH₃ | A |
| | 1235 | 417, 981, 1091, 1235 | Aquaterin V | C₆₂H₁₀₈O₂₄ | Octa | H | H | Octa | Rha | CH₃ | A |
| 19.8 | 1089 | 417, 543, 691, 819, 1089 | Aquaterin XI | C₅₆H₉₈O₂₀ | Octa | H | H | H | Octa | CH₃ | A |
| 20.3 | 1193 | 417, 543, 939, 1049, 1193 | Aquaterin III | C₅₉H₁₀₂O₂₄ | Octa | H | H | Mba | Rha | CH₃ | B |
| | 1193 | 417, 543, 939, 1049, 1193 | Aquaterin II | C₅₉H₁₀₂O₂₄ | Octa | H | H | Mba | Rha | CH₃ | A |
| | 1193 | 417, 543, 939, 1049, 1193 | Murucoidin XVIII | C₅₉H₁₀₂O₂₄ | Mba | H | H | Octa | Rha | CH₃ | B |
| | 1193 | 417, 543, 939, 1049, 1193 | Stoloniferin VIII | C₅₉H₁₀₂O₂₄ | Mba | H | H | Octa | Rha | CH₃ | A |
| 20.3 | 1195 | 417, 543, 647, 757, 837, 939, 1049, 1195 | Formula (IV) | C₅₈H₁₀₀O₂₅ | Iba | H | H | Octa | Glu | CH₃ | A |
| 21.8 | 1299 | 417, 543, 693, 963, 1107, 1209, 1299 | Formula (V) | C₆₈H₁₂₀O₂₂ | Octa | Octa | H | Mba | Octa | CH₃ | A |
| 22.5 | 1117 | 417, 571, 691, 837, 991, 1117 | Formula (VI) | C₅₈H₁₀₂O₂₀ | Deca | H | H | H | Octa | CH₃ | A |
| 40.3 | 1319 | 417, 543, 921, 1065, 1175, 1319 | Aquaterin XIV | C₆₇H₁₁₆O₂₅ | Octa | H | Octa | Mba | Rha | CH₃ | A |
| | 1319 | 417, 543, 921, 1065, 1175, 1319 | Aquaterin XIII | C₆₇H₁₁₆O₂₅ | Octa | Octa | H | Mba | Rha | CH₃ | B |
| | 1319 | 417, 543, 921, 1065, 1175, 1319 | Aquaterin XII | C₆₇H₁₁₆O₂₅ | Octa | H | Octa | Mba | Rha | CH₃ | B |
| 40.8 | 1335 | 417, 543, 754, 935, 1089, 1191, 1335 | Formula (VII) | C₆₇H₁₁₆O₂₆ | Octa | H | Octa | Mba | Glu | CH₃ | A |

*Mba: 2-methylbutanoyl, Dodeca: n-dodecanoyl, Octa: n-octanoyl, Deca: n-decanoyl, Cna: cinnamoyl, Rha: Rhamnopyranosyl, Glu: Glucopyranosyl; Iba: Isobutanoyl

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. An extract comprising at least one resin glycoside selected from the group consisting of Batataoside I, Batataoside II, Batatoside III, Pescaprein XXVII, Batatoside F, Batatoside H, a resin glycoside of Formula (I), 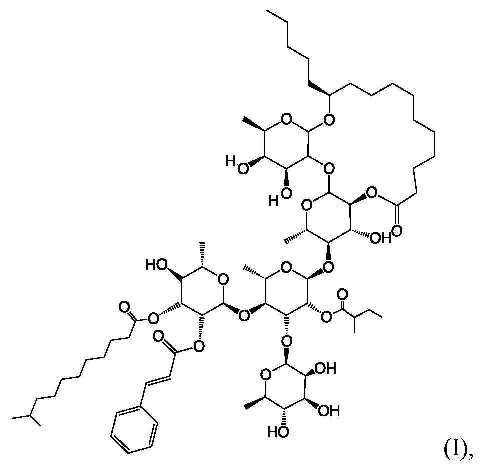
a resin glycoside of Formula (II) 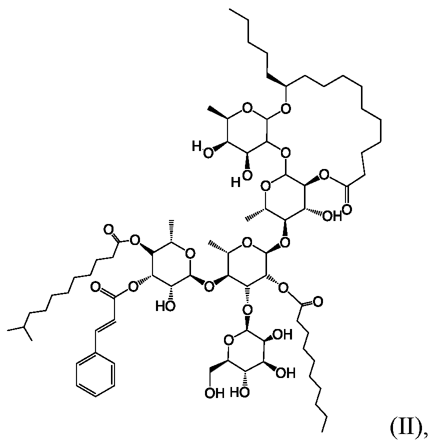
wherein the resin glycoside is extracted from a plant selected from Ipomoea batatas (sweet potato), and/or
comprising at least one resin glycoside selected from the group consisting of Batatoside F, Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII), 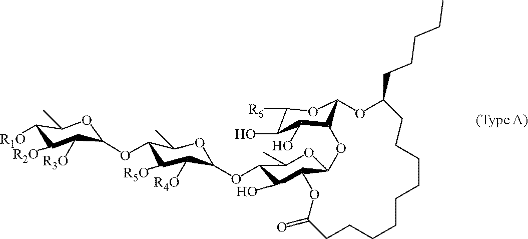
wherein Formula (III) is when R₁ is n-dodecanoyl, R₂ is cinnamoyl, R₃ is 2-methylbutanoyl, R₄ is H, R₅ is rhamnopyranosyl, and R₆ is methyl,
wherein Formula (IV) is when R₁ is isobutanoyl, R₂ is H, R₃ is H, R₄ is n-octanoyl, R₅ is glucopyranosyl, and R₆ is methyl,
wherein Formula (V) is when R₁ is n-octanoyl, R₂ is n-octanoyl, R₃ is H, R₄ is 2-methylbutanoyl, R₅ is n-octanoyl, and R₆ is methyl,
wherein Formula (VI) is when R₁ is n-dodecanoyl, R₂ is H, R₃ is H, R₄ is H, R₅ is n-octanoyl, and R₆ is methyl,
wherein Formula (VII) is when R₁ is n-octanoyl, R₂ is H, R₃ is n-octanoyl, R₄ is 2-methylbutanoyl, R₅ is glucopyranosyl, and R₆ is methyl, and
wherein the resin glycoside is extracted from a plant selected from Ipomoea aquatica (kangkong).

2. The extract according to claim 1, wherein when Batataoside I is present, the concentration is about 0.5 mg/g to about 500 mg/g relative to the extract;
wherein when Batataoside II is present, the concentration is about 0.1 mg/g to about 5 mg/g relative to the extract;
wherein when Batataoside III is present, the concentration is about 0.1 mg/g to about 10 mg/g relative to the extract;
wherein when Batatoside F is present, the concentration is about 2 mg/g to about 40 mg/g relative to the extract;
wherein when Batatoside H is present, the concentration is about 2 mg/g to about 8 mg/g relative to the extract;
wherein when Pescaprein XXVII is present, the concentration is about 0.1 mg/g to about 5 mg/g relative to the extract;
wherein when resin glycoside of Formula (I) is present, the concentration is about 0.1 mg/g to about 4 mg/g relative to the extract; and
when resin glycoside of Formula (II) is present, the concentration is about 10 mg/g to about 500 mg/g relative to the extract.

3. The extract according to claim 1, comprising a mixture of at least 2 resin glycosides.

4. The extract according to claim 1, wherein the at least one resin glycoside is Batatoside I and resin glycoside of Formula (I).

5. The extract according to claim 1, having a lipase inhibition activity IC₅₀ value of less than 15 µg/mL.

6. (canceled)

7. An edible composition, comprising:
at least one resin glycoside selected from the group consisting of Batataoside I, Batataoside II, Batatoside III, Pescaprein XXVII, Batatoside F, Batatoside H, a resin glycoside of Formula (I), a resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea batatas (sweet potato) and/or Ipomoea aquatica (kangkong).

8. The edible composition according to claim 7, further comprising a component selected from protein, fiber, polyphenolic compound, lipid or a combination thereof.

9. The edible composition according to claim 7, the edible composition is provided as a capsule.

10. The edible composition according to claim 7, for use in weight loss or weight management.

11. (canceled)

12. A method of extracting at least one resin glycoside from a plant selected from the Convolvulaceae family, comprising:
a) subjecting the plant to a solvent extraction process for obtaining a crude extract;
b) purifying the crude extract for obtaining a partially purified extract; and
c) further purifying the partially purified extract for obtaining the resin glycoside
wherein the resin glycoside is selected from the group consisting of Batataoside I, Batataoside II, Batatoside III, Pescaprein XXVII, Batatoside F, Batatoside H, a resin glycoside of Formula (I), a resin glycoside of Formula (II), Batatoside E, Batatoside D, Aquaterin VI, Aquaterin V, Aquaterin XI, Aquaterin II, Stoloniferin VIII, Aquaterin XIV, Batatoside C, Batatinoside I, Aquaterin VII, Aquaterin III, Murucoidin XVIII, Aquaterin XIII, Aquaterin XII, a Type A resin glycoside of Formula (III), Formula (IV), Formula (V), Formula (VI), and Formula (VII);
wherein the resin glycoside is extracted from a plant selected from Ipomoea batatas (sweet potato) and/or Ipomoea aquatica (kangkong).

13. The method according to claim 12, wherein the resin glycoside is extracted from a tuber, leaf, latex, and stem of Ipomoea batatas (sweet potato), or from a seed, leaf or stem of Ipomoea aquatica (kangkong).

14. The method according to claim 12, wherein the step of subjecting the plant to a solvent extraction process (step a) comprises homogenising the plant in a solvent and liquid-liquid extracting the resin glycoside as a crude extract.

15. The method according to claim 12, wherein the step of purifying the crude extract (step b) comprises fractionating the crude extract.

16. The method according to claim 12, wherein the step of purifying the crude extract (step b) comprises eluting the crude extract through a liquid chromatography column with a predetermined mobile phase, wherein the mobile phase is selected from:
a) a gradient of hexane:ethyl acetate (hexane:EtOAc: 1:1) to pure ethyl acetate, followed by pure methanol;
b) a gradient of dichloromethane:methanol (DCM:MeOH: 7:1) to pure MeOH; or
c) a gradient of H₂O:MeOH (100:0) to pure MeOH.

17. (canceled)

18. (canceled)

19. (canceled)

20. The method according to claim 12, wherein the further purifying step (step c) comprises fractionating the partially purified extract.

21. The method according to claim 12, wherein the further purifying step (step c) comprises eluting the partially purified extract through a liquid chromatography column with a predetermined mobile phase.

22. The method according to claim 12, wherein the further purifying step (step c) comprises eluting the partially purified extract through a liquid chromatography column with a mobile phase having a gradient of MeOH-H₂O (90:10) to MeOH-H₂O (100:0).

23. The method according to claim 12, wherein the further purifying step (step c) further comprises eluting a faction from the liquid chromatography column with a mobile phase having a gradient of MeOH-H₂O (90:10) to MeOH-H₂O (100:0) with acetonitrile or with ACN-MeOH (80:20).

24. A method of treating a disease or disorder associated with excessive body fat in a subject in need thereof, comprising administering resin glycoside from a plant selected from the Convolvulaceae family.

25. The method according to claim 24, wherein the disease or disorder is selected from obesity overweight, and metabolic syndrome.
